(19) United States
(12) Patent Application Publication
(10) Pub. No.: US 2018/0110810 A1
(43) Pub. Date: Apr. 26, 2018

(54) FREEZE DRIED FECAL MICROBIOTA FOR USE IN FECAL MICROBIAL TRANSPLANTATION

(71) Applicant: Regents of the University of Minnesota, Minneapolis, MN (US)

(72) Inventors: Michael J. Sadowsky, Roseville, MN (US); Alexander Khoruts, Golden Valley, MN (US); Matthew James Hamilton, Burnsville, MN (US); Aleh Bobr, Rochester, MN (US); Alexa Rachel Weingarden, St. Paul, MN (US)

(21) Appl. No.: 15/837,834

(22) Filed: Dec. 11, 2017

Related U.S. Application Data
(63) Continuation of application No. 14/850,318, filed on Sep. 10, 2015, now abandoned.
Application No. 14/850,318 is a continuation-in-part of application No. PCT/US2014/027391, filed on Mar. 14, 2014.
Application No. 14/850,318 is a continuation-in-part of application No. 14/003,411, filed on Jan. 17, 2014.
(60) Provisional application No. 61/782,222, filed on Mar. 14, 2013.
(60) Provisional application No. 61/450,838, filed on Mar. 9, 2011.

(51) Int. Cl. A61K 35/741; A61K 35/76; A61K 35/74

(52) U.S. Cl. CPC A61K 35/741; A61K 35/76; A61K 35/74

(57) ABSTRACT

The present invention provides freeze-dried compositions that include an extract of human feces and a cryoprotectant, and methods for making and using such compositions, including methods for replacing or supplementing or modifying a subject's colon microbiota, and methods for treating a disease, pathological condition, and/or iatrogenic condition of the colon.

### CROSS-REFERENCE TO RELATED APPLICATIONS

This application is a continuation-in-part of International Application Serial No. PCT/US2014/027391, filed on Mar. 14, 2014, which claims the benefit of U.S. Provisional Application Ser. No. 61/782,222, filed Mar. 14, 2013. This application is also a continuation-in-part of U.S. application Ser. No. 14/003,411, filed on Jan. 17, 2014, which is a U.S. National Stage Application of International Application Serial No. PCT/US2012/028484, filed on Mar. 9, 2012, which claims the benefit of U.S. Provisional Application Ser. No. 61/450,838, filed Mar. 9, 2011. All of the foregoing applications are incorporated by reference in their entirety.

### GOVERNMENT FUNDING

This invention was made with government support under R21AI091907 awarded by the National Institutes of Health. The government has certain rights in the invention.

### BACKGROUND

In 1978, Clostridium difficile was first recognized as a major cause of diarrhea and pseudomembranous colitis associated with the use of antimicrobial agents. Since this time, infection by C. difficile has been steadily growing in incidence, morbidity, and mortality across North America and Europe (Freeman et al. Clin Microbiol Rev 2010; 23:529-49, Kelly and LaMont. N Engl J Med 2008; 359:1932-40). Analysis of the U.S. National Hospital Discharge Survey statistics between 1996 and 2003 reveals a doubling in the prevalence of diagnosis of C. difficile infection (CDI), to 0.61/1,000, among inpatients (McDonald et al. Emerg Infect Dis 2006; 12:409-15). A 2008 survey of 12.5% of all U.S. acute care facilities indicated a CDI prevalence rate of 13.1/1,000, which is at least an order of magnitude higher than that found previously (Jarvis et al. Am J Infect Control 2009; 37:263-70). While older patients have disproportionately greater rates of CDI than younger individuals, no age group is spared, and the incidence of CDI-related hospitalizations has been rising even in the pediatric population (Zilberberg et al. Emerg Infect Dis 2010; 16:604-9). The increase in incidence has been further compounded by an elevated frequency of the most severe forms of this disease, as evidenced by rising CDI-associated morbidity and case fatality (Ricciardi et al. Arch Surg 2007; 142:624-31; discussion 631, Zilberberg et al. Emerg Infect Dis 2008; 14:92931). This is, in part, related to the emergence of more virulent C. difficile strains, such as PCR ribotype 027/North American Pulsed Field type 1 (NAP1), which is characterized by a greater potential for toxin production and antibiotic resistance than other clinically-relevant rains (Rupnik et al. Nat Rev Microbiol 2009; 7:526-36, Kuijper et al. Euro Surveill 2008; 13).

Recurrent CDI is one of the most difficult and increasingly common challenges associated with CDI (Surawicz, Gastroenterology 2009; 136:1152-4). An initial incidence of CDI can be followed by a relapse within 30 days in about 20-30% of cases (Kelly and LaMont. N Engl J Med 2008; 359:1932-40, Louie et al. N Engl J Med 2011; 364:422-31, Pepin et al. Clin Infect Dis 2006; 42:758-64), and the risk of recurrence doubles after two or more occurrences (McDonald et al. Emerg Infect Dis 2006; 12:40915). Older age, intercurrent antibiotic use for non-C. difficile indications, renal insufficiency, immune deficiency, and antacid medications, are some of the known risk factors for recurrent CDI (Surawicz, Gastroenterology 2009; 136:1152-4, Garey et al. J Hosp Infect 2008; 70:298-304). The presence of just three clinical criteria: age>65 years, severe disease, and continued use of antibiotics after treating the initial CDI episode, are predictive of an almost 90% relapse rate (Hu et al. Gastroenterology 2009; 136:1206-14). CDI also commonly complicates management of inflammatory bowel disease (IBD), which has recently been recognized as an additional independent risk factor for CDI infection (Issa et al. Clin Gastroenterol Hepatol 2007; 5:345-51, Rodemann et al. Clin Gastroenterol Hepatol 2007; 5:339-4415). CDI in patients with underlying IBD is associated with increased severity of colitis and higher rates of recurrence and colectomy (Issa et al. Inflamm Bowel Dis 2008; 14:1432-42).

It is now recognized that the presence of normal, healthy, intestinal microbiota (normal gut microorganisms) offers protection against CDI. Conversely, severe disruption of normal intestinal microbiota by use of antibiotics, including metronidazole and vancomycin that are used to treat CDI, is likely one of the major reason for its recurrence. Chang and colleagues used 16S rDNA sequencing to analyze the fecal microbiota of seven patients with initial and recurrent CDI (Chang et al. J Infect Dis 2008; 197:435-8). They reported that bacterial species diversity was reduced in all patients compared to nomial control subjects. The greatest reduction in species diversity, however, was found in the three patients with recurrent CDI and disruption of their gut microbiota was evident at the phylum level—with marked reduction in Bacteriodetes, normally one of the two dominant phyla in the colon. Instead, the gut microbiota in these patients were dominated by members of the proteobacteria and verrucomicrobia phyla, which usually are only minor constituents of the colon microbiota.

The general aim of antibiotic treatment for recurrent CDI is not mere suppression of C. difficile, but also preservation of the residual colon microbiota and optimization of their restoration. Various antibiotic regimens, including long tapered or pulsed dosing with vancomycin (McFarland et al. Am J Gastroenterol 2002; 97:1769-75) and rifaximin “chaser” protocols (Johnson et al. Clin Infect Dis 2007; 44:846-8, Johnson et al. Anaerobe 2009; 15:290-1) have been used to achieve this objective with partial success. Recently, fidaxomicin, a new macrocyclic antibiotic which is narrow in spectrum and spares Bacteroides species, was shown to reduce the initial relapse rate of CDI by 50% compared to vancomycin treatment (Louie et al. N Engl J Med 2011; 364:422-31). However, treatment with fidaxomicin did not alter the recurrence rate of CDI caused by the more virulent PCR 027/NAP1 strain. Therefore, despite these advances it seems likely that the challenges in treatment of recurrent CDI will remain for the foreseeable future.

Fecal microbiota transplantation (FMT), also commonly known as ‘fecal bacteriotherapy,’ represents the one therapeutic protocol that allows the fastest reconstitution of a normal composition and functional gut microbial community. For many decades, FMT has been offered by select centers across the world, typically as an option of last resort for patients with recurrent Clostridium difficile infection (CDI). The mostly commonly earliest cited report for FMT was by Eiseman and colleagues who in 1958 described the use of fecal enemas for patients who likely had severe or fulminant form of pseudomembranous colitis (Eiseman et al. Surgery 1958; 44:854-9). Since this time, well over 500 cases have been reported as individual case reports, small case series, or clinical trials with a ˜90% cumulative success rate in clearing recurrent CDI, without any noted adverse events. The history and general methodology used for FMT have been described in several recent reviews (Bakken. Anaerobe 2009; 15:285-9, van Nood et al. Euro Surveill 2009; 14, Khoruts and Sadowsky. Mucosal Immunol 2011; 4:4-7). A recent randomized, controlled clinical study has confirmed the remarkable efficacy of this therapeutic approach (van Nood et al., 2013, N Engl J Med, 368:407-15). However, despite the long and successful track record, as well as great clinical need, the availability of the procedure for many patients remains very limited.

The lack of wider practice of FMT is due, in large part, to multiple non-trivial practical barriers and not due to lack of efficacy. These include lack of reimbursement for donor screening, lack of insurance coverage, lack of adequate donors at the correct time, difficulty in material preparation and administration, as well as aesthetic concerns about doing the procedure in endoscopy or medical office. These also include patient perception of the procedure, willingness of staff to perform the procedure, sanitation issues related to manipulation of fecal matter, and the odiferous nature of fecal slurries. Together these factors make it a distasteful option that is often considered a treatment of last resort, and that is largely unavailable to the vast majority of patients who could benefit from it. Moreover, the pharmaceutical industry has shown little interest in technological development of FMT-based therapeutics, in large part due to the wide availability of donor material and its complex composition. Instead, development has been driven mostly by individual clinicians faced with desperate needs of their patients.

### SUMMARY OF THE APPLICATION

The present invention provides compositions that include an extract or a preparation of human feces. In one embodiment, a composition includes no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. Optionally the biological material includes human gut, colon or intestinal fecal microbes, and optionally the biological material includes human gut, colon or intestinal bacteria. Optionally the composition includes a pharmaceutically acceptable carrier. Optionally the composition is a formulation for oral administration.

In one embodiment, a composition consists of, or consists essentially of, particles of non-living material and/or particles of biological material that will pass through a sieve having a sieve size of 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.2 mm. Optionally the composition includes a pharmaceutically acceptable carrier, and optionally the composition is a formulation for oral administration.

In one embodiment, a composition includes at least 4 different phyla of gut, colon or intestinal bacteria extracted or prepared from the gut, colon or intestine, wherein the phyla include a member of Bacteroidetes phylum, member of Firmicutes phylum, member of Proteobacteria phylum, member of Tenericutes phylum, or a combination thereof. Optionally the phyla are chosen from Bacteroidetes, Firmicutes, Proteobacteria, and Tenericutes. The composition includes no greater than 0.05%, 0.1%, 0.2%, 0.3%, 0.4%, 0.5%, 0.6%, 0.7%, 0.8%, 0.9% 1%, 2%, 3%, 4%, 5%, 6%, 7%, 8%, 9%, or 10% weight non-living material/weight biological material. Optionally the biological material includes human gut, colon or intestinal flora. Optionally the biological material includes human gut, colon or intestinal bacteria. Optionally the composition includes a pharmaceutically acceptable carrier, and optionally the composition is a formulation for oral administration.

In one embodiment, a composition includes an extract of human feces wherein the composition is substantially odorless, optionally includes biological material, and optionally wherein the biological material includes bacteria. Optionally the composition includes a pharmaceutically acceptable carrier, and optionally the composition is a formulation for oral administration.

A composition of the present invention may include no greater than 0.1% weight non-living material/weight biological material. In one embodiment, a composition may consist of, or consist essentially of, particles that will pass through a 0.25 mm sieve, or equivalent. In one embodiment, a composition may include no greater than 0.05%, 0.1%, 0.2%, 0.3%, 0.4%, 0.5%, 0.6%, 0.7%, 0.8%, 0.9%, 1%, 2%, 3%, 4% 5%, 6%, 7%, 8%, 9% or 10% weight non-living material/weight biological material. A composition of the present invention may further include a cryoprotectant, such as glycerol. In one embodiment, a composition may be at a temperature of less than 0° C. In one embodiment, a composition is a solid, such as a powder. A composition of the present invention may include at least 1×10¹⁰, 2×10¹⁰, 3×10¹⁰, 4×10¹⁰, or 5×10¹⁰ bacteria. In one embodiment, the biological material of a composition may include a plurality of prokaryotic cells, eukaryotic cells, or viruses; or a population of prokaryotic cells, eukaryotic cells, and viruses, that is substantially identical to or representative of or equivalent to a population of prokaryotic cells, eukaryotic cells, and viruses present in a feces of a normal healthy human. In one embodiment, the biological material of a composition may include a population of prokaryotic cells and viruses that is substantially identical to or representative of or equivalent to a population of prokaryotic cells and viruses present in the feces of a normal healthy human. In one embodiment, the biological material of a composition includes a population of prokaryotic cells, eukaryotic cells, or viruses that is substantially identical to or representative of or equivalent to a population of prokaryotic cells, eukaryotic cells, and viruses present in the feces of a normal healthy human.

The present invention also provides composition prepared by a process. In one embodiment, a process includes subjecting a fecal sample to a condition or conditions that remove at least 91%, 92%, 93%, 94% 95%, 96%, 97%, 98%, 99% or more of the non-living material present in the fecal sample. In one embodiment, a process includes filtering a fecal sample with a filter medium, wherein the filter medium includes a sieve size of no greater than 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.2 mm to result in or to generate a filtrate. Optionally a composition includes a biological material, and optionally the biological material includes bacteria. Optionally a composition includes a pharmaceutically acceptable carrier. Optionally a composition is a formulation for oral administration. Optionally the process may occur at a temperature of no greater than 26° C., 27° C., 28° C., 29° C., 30° C., 31° C., 32° C., 33° C., or 34° C.

The composition may include at least 4 different phyla of bacteria, wherein the include a member of Bacteroidetes phylum, member of Firmicutes phylum, member of Proteobacteria phylum, member of Tenericutes phylum, or a combination thereof Optionally the phyla are chosen from Bacteroidetes, Firmicutes, Proteobacteria, and Tenericutes. In one embodiment, the composition further includes at least 5, 6, 7, 8, 9, or 10 different classes of bacteria chosen from Actinobacteria, Bacteroidia, Bacilli, Clostridia, Erysipelotrichi, Alphaproteobacteria, Betaproteobacteria, Gammaproteobacteria, Mollicutes, and Verrucomicrobiae.

The process may further include adding a cryoprotectant, for instance, glycerol, to the composition. The process may further include freezing the composition. The composition may be for use as a therapeutic agent, and it may be for use in the treatment of a disease or a pathological or iatrogenic condition of the colon. The disease may be a disease or condition characterized by a dysfunctional or pathological composition of colon microbiota, for instance, a Clostridium difficile colitis.

The present invention also provides a method for replacing or supplementing or modifying a subject's colon microbiota. The method may include administering to the subject a composition described herein. The present invention also provides a method for treating a subject. The method may include administering to a subject in need thereof an effective amount of a composition described herein. The methods may further include removal of some, most, or substantially all of the subject's colon, gut or intestinal microbiota prior to the administering. The subject may have or be at risk for having a colitis. In one embodiment, the colitis is an autoimmune colitis, such as an inflammatory bowel disease, an ulcerative colitis, a Crohn's disease, or an irritable bowel syndrome. In one embodiment, the colitis is an infectious colitis, such as a Clostridium difficile colitis or an enterohemorrhagic colitis. The Clostridium difficile colitis may be an acute Clostridium difficile colitis, a relapsing Clostridium difficile colitis, and a severe Clostridium difficile colitis. The enterohemorrhagic colitis may be caused by a Shigella spp. or an E. coli. The subject may have or be at risk for chronic diarrhea or chronic constipation.

The freeze-dried compositions presented herein provide a significant advantage by making useful compositions of intestinal microflora, for instance, colon microflora. Such compositions may be readily available for use by a physician to treat a patient, readily available for use by a patient at home, and/or readily available as an over-the-counter composition for sale directly to a consumer. In some embodiments, the compositions described herein provide advantages including, better long term storage, better transport, and options regarding delivery to the patient. In one embodiment, delivery is by oral administration of a capsule that contains a freeze-dried composition. In another embodiment, the material can be delivered via nasogastric tube, enema, or colonoscopy.

Provided herein are freeze-dried compositions. In one embodiment, the composition includes biological material and a cryoprotectant, wherein the freeze-dried composition is friable, wherein optionally the composition includes a pharmaceutically acceptable carrier, and optionally the composition is a formulation for oral administration, and wherein the freeze-dried composition, upon reconstitution with water, includes no greater than about 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% weight non-living material/weight biological material, wherein the biological material includes human gut, colon or intestinal fecal microbes, and optionally the biological material includes human gut, colon or intestinal bacteria.

In one embodiment, the composition includes an extract or preparation of human feces including human fecal material and a cryoprotectant, wherein the freeze-dried composition is friable, wherein the human fecal material, upon reconstitution with water, consists of, or consists essentially of, particles of non-living material and/or particles of biological material that will pass through a sieve having a sieve size of 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, or 0.01 mm, and wherein optionally the composition includes a pharmaceutically acceptable carrier, and optionally the composition is a formulation for oral administration.

In one embodiment, the composition includes at least 4 different phyla of gut, colon or intestinal bacteria extracted or prepared from the gut, colon or intestine, and a cryoprotectant, wherein the phyla include a Bacteroidetes, a Firmicutes, a Proteobacteria a Tenericutes phylum, or a combination thereof, wherein optionally the phyla are chosen from Bacteroidetes, Firmicutes, Proteobacteria, Tenericutes, or a combination thereof, wherein the composition, upon reconstitution with water, includes no greater than about 0.05%, 0.1%, 0.2%, 0.3%, 0.4%, 0.5%, 0.6%, 0.7%, 0.8%, 0.9% 1%, 2%, 3%, 4%, 5%, 6%, 7%, 8%, 9% or 10% weight non-living material/weight biological material, wherein the biological material includes human gut, colon or intestinal fecal microbes, and optionally the biological material includes human gut, colon or intestinal bacteria, and wherein optionally the composition includes a pharmaceutically acceptable carrier, and optionally the composition is a formulation for oral administration.

In one embodiment, the composition includes an extract of human feces and a cryoprotectant, wherein the composition, upon reconstitution with water, is substantially odorless, wherein the composition includes biological material, and optionally wherein the biological material includes microbes, and wherein optionally the composition includes a pharmaceutically acceptable carrier, and optionally the composition is a formulation for oral administration.

In one embodiment, the fecal material consists of, or consists essentially of, particles that will pass through a 0.25 mm sieve, or equivalent. In one embodiment, the composition includes at least about 1×10¹², 1.5×10¹², 2×10¹², or 2.5×10¹² bacteria.

In one embodiment, the biological material includes: a plurality of prokaryotic cells, eukaryotic cells, or viruses; or a population of prokaryotic cells, eukaryotic cells, and viruses, that is substantially identical to or representative of or equivalent to a population of prokaryotic cells, eukaryotic cells, and viruses present in gut, intestine, colon, or feces of a normal healthy human. In one embodiment, the biological material present includes a population of prokaryotic cells and viruses that is substantially identical to or representative of or equivalent to a population of prokaryotic cells and viruses present in the feces of a normal healthy human. In one embodiment, the biological material includes a population of prokaryotic cells, eukaryotic cells, or viruses that is substantially identical to or representative of or equivalent to a population of prokaryotic cells, eukaryotic cells, and viruses present in the feces of a normal healthy human.

In some embodiments the compositions are prepared by a process. In one embodiment, the process includes subjecting a fecal sample to a condition or conditions that removes at least about 91%, 92%, 93%, 94% 95%, 96%, 97%, 98%, 99% or more of the non-living material present in the fecal sample prior to the subjecting to result in an extract, adding a cryoprotectant to the extract to result in a mixture, and freeze-drying the mixture to result in the composition. The composition includes a biological material, and optionally the biological material includes bacteria, wherein optionally the composition includes a pharmaceutically acceptable carrier, and optionally the composition is a formulation for oral administration. In one embodiment, the subjecting occurs at a temperature of no greater than about 26° C., 27° C., 28° C., 29° C., 30° C., 31° C., 32° C., 33° C., or 34° C.

In one embodiment, the process includes filtering a fecal sample with a filter medium, wherein the filter medium includes at least one sieve size of no greater than about 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, or 0.01 mm to result in or to generate a filtrate, adding a cryoprotectant to the filtrate to result in a mixture, freeze-drying the mixture to result in a freeze-dried composition, and milling the freeze-dried composition into a powder. The composition includes a biological material, and optionally the biological material includes bacteria, wherein optionally the composition includes a pharmaceutically acceptable carrier, and optionally the composition is a formulation for oral administration.

In one embodiment, the process further includes reconstituting the composition with an aqueous solution. In one embodiment, the process further includes filtering the fecal sample with a filter medium, wherein the filter medium includes a sieve size of no greater than 0.25 mm. In one embodiment, the filtering occurs at a temperature of no greater than about 26° C., 27° C., 28° C., 29° C., 30° C., 31° C., 32° C., 33° C., or 34° C.

In one embodiment, the composition includes at least 4 different phyla of bacteria, wherein the phyla include a Bacteroidetes, a Firmicutes, a Proteobacteria, a Tenericutes phyla, or a combination thereof, wherein optionally the phyla are chosen from Bacteroidetes, Firmicutes, Proteobacteria, Tenericutes, or a combination thereof. In one embodiment, the composition further includes at least 5, 6, 7, 8, 9, or 10 different classes of bacteria chosen from Actinobacteria, Bacteroidia, Bacilli, Clostridia, Erysipelotrichi, Alphaproteobacteria, Betaproteobacteria, Gammaproteobacteria, Mollicutes, and Verrucomicrobiae.

In one embodiment, the cryoprotectant present in a composition described herein includes skim milk, gelatin, mannitol, or a combination thereof. In one embodiment, a composition described herein includes at least two cryoprotectants, wherein the first cryoprotectant is sucrose. In one embodiment, a second cryoprotectant is selected from skim milk, gelatin, and mannitol. In one embodiment, the cryoprotectant is present at a concentration of at least 1%, 2%, 3%, 4%, 5%, 6%, 7%, 8%, 9%, or 10% (vol/vol). In one embodiment, the cryoprotectant of a composition described herein does not include glycerol. In one embodiment, a composition described herein does not include surcose as the only cryoprotectant.

In one embodiment, a composition described herein is encapsulated in a capsule. In one embodiment, the capsule includes an acid-resistant enteric coating.

In one embodiment, a composition described herein is for use as a therapeutic agent. In one embodiment, a composition described herein is for use in the treatment of a disease or a pathological or iatrogenic condition of the colon. In one embodiment, the disease is a disease or condition characterized by a dysfunctional or pathological composition of colon microbiota. In one embodiment, the disease is a Clostridium difficile colitis, such as acute Clostridium difficile colitis, relapsing Clostridium difficile colitis, or severe Clostridium difficile colitis.

Also provided herein are methods. In one embodiment, the method is for replacing or supplementing or modifying a subject's colon microbiota. In one embodiment, such a method includes administering to the subject the composition described herein. In one embodiment, the method further includes reconstituting the composition with an aqueous solution.

In one embodiment, the method is for treating a subject. In one embodiment, such a method includes administering to a subject in need thereof an effective amount of a composition described herein. In one embodiment, the subject has or is at risk for having a colitis, such as an autoimmune colitis. In one embodiment, the autoimmune colitis is selected from an inflammatory bowel disease, an ulcerative colitis, a Crohn's disease and an irritable bowel syndrome. In one embodiment, the colitis is an infectious colitis. In one embodiment, the infectious colitis is selected from a Clostridium difficile colitis and an enterohemorrhagic colitis. In one embodiment, the Clostridium difficile colitis is selected from an acute Clostridium difficile colitis, a relapsing Clostridium difficile colitis, and a severe Clostridium difficile colitis. In one embodiment, the enterohemorrhagic colitis is caused by a microbe selected from a Shigella spp. and an E. coli. In one embodiment, the subject has or is at risk for chronic diarrhea or chronic constipation.

In one embodiment, a method further includes removal of some, most, or substantially all of the subject's colon, gut or intestinal microbiota prior to the administering.

Further provided a uses of a composition described herein for the manufacture of a medicament. In one embodiment, the use of a composition is for the manufacture of a medicament for treating or ameliorating or preventing a disease or a pathological or iatrogenic condition of the colon, wherein optionally the disease is a disease or condition characterized by a dysfunctional or pathological composition of colon microbiota, or the disease is a Clostridium difficile colitis, or the disease or condition is a colitis, an autoimmune colitis, an infectious colitis or an enterohemorrhagic colitis.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

Before the present invention standard practices suggested matching each recipient of fecal bacteriotherapy with a separate donor, usually a close family member, or using the recipient's own banked feces for later use. The rationale for these practices was the idea that close family members have already shared their pathogens, and that these kinds of gut microbiota would be somehow better tolerated by the recipient's immune system because of previous exposure. However, this resulted in duplicative screening, burdening already debilitated patients with the task of finding a suitable donor, pressure on the donor to provide the material and potentially withholding important medical information, pressure to decrease costs since costs were usually borne by the patient, time delays associated with the screening, and pressure to accept donors of suboptimal health status during donor selection. The compositions presented herein result from a more standardized manufacturing process with rigorous donor screening, multiple steps of filtration that concentrate the microbiota and remove the bulk of nonliving material, and optionally freeze/thaw it in a way that preserves its viability. The compositions presented herein provide a significant advantage by making useful compositions of colon microflora readily available for use by a physician to treat a patient. Moreover, it is much more aesthetically acceptable, as the compositions are nearly odorless, are in concentrated form, and are easily manipulated using standard laboratory practice.

Provided herein are freeze-dried compositions that include fecal microbes. As used herein, the term “fecal microbes” refers to microorganisms that are present in the gut, intestine, or colon, preferably colon, or feces of a normal healthy adult human. Such a freeze-dried composition may be prepared by processing fecal material. As used herein, the term “fecal material” refers to human stool. Unprocessed fecal material contains non-living material and biological material. The “non-living material” refers to the non-living material in fecal material, and may include, but is not limited to, dead bacteria, shed host cells, proteins, carbohydrates, fats, minerals, mucus, bile, undigested fiber and other foods, and other compounds resulting from food and metabolic ingestion and waste products and partial or complete digestion of food materials. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archea (e.g., living prokaryotic cells and spores that can sporulate to become living prokaryotic cells), eukaryotic cells such as protozoa and fungi, and viruses. In one embodiment, “biological material” refers to the living material, e.g., the microbes, eukaryotic cells, and viruses, which are present in the colon of a normal healthy human.

As used herein, “freeze-dried” refers to a composition having the characteristics described herein and further having substantially no water present, and in one embodiment, no detectable water. Methods for freeze-drying a composition are known and routinely used. The word freeze-drying is used synonymously with lyophilization. A method for freeze-drying a composition may include one or more pretreatments (e.g., concentrating, addition of a cryoprotectant, increasing the surface area of a composition), freezing the composition, and drying (e.g., exposing the composition to a reduced atmospheric pressure to result in sublimation of the water present in the composition).

Examples of prokaryotic cells that may be present in a freeze-dried composition described herein include cells that are members of the class Actinobacteria, such as the subclass Actinobacteridae and subclass Coriobacteridae. Examples of the subclass Actinobacteridae include members of the order Actinomycetales, and members of the order Bifidobacteriales. Members of the order Bifidobacteriales include members of the family Bifidobacteriaceae. Examples of the subclass Coriobacteridae include members of the order Coriobacteriales. Members of the order Coriobacteriales include members of the family Coriobacteriaceae.

Other examples of prokaryotic cells include members of the phylum Bacteroidetes, such as class Bacteroidia. Members of class Bacteroidia include order Bacteroidales. Members of order Bacteroidales include members of the family Bacteroidaceae, members of the family Porphyromonadaceae, members of the family Prevotellaceae, and members of the family Rikenellaceae.

Other examples of prokaryotic cells include members of the phylum Firmicutes, such as class Bacilli, Clostridia, Erysipelotrichi, and Negativicutes. Examples of the class Bacilli include members of the order Bacillales (including members of the family Paenibacillaceae and members of the family Planococcaceae) and the order Lactobacillales (including members of the family Aerococcaceae, Enterococcaceae, Lactobacillaceae, and Streptococcaceae). Examples of the class Clostridia include members of the order Clostridiales, and examples of the order Colstridiales include the family Catabacteriaceae, Peptococcaceae, Peptostreptococcaceae, Ruminococcaceae, Clostridiaceae, Eubacteriaceae, and Lachnospiraceae. Examples of the class Erysipelotrichi include members of the family Erysipelotrichaceae. Examples of the class Negativicutes include members of the family Veillonellaceae. Other examples of the order Bacillales include Bacillales Family XI._Incertae Sedis, and Bacillaceae 1. Other examples of the order Clostridiales include Clostridiales Family XI. Incertae Sedis, Clostridiales Family XIII. Incertae Sedis, and Clostridiaceae.

Other examples of prokaryotic cells include members of the phylum Proteobacteria, such as class Alphaproteobacteria, Betaproteobacteria, Deltaproteobacteria, Epsilonproteobacteria, and Gammaproteobacteria. Examples of the class Alphaproteobacteria include members of the order Rhizobiales, and examples of members of the order Rhizobiales includes members of the family Rhodobiaceae, members of the family Brucellaceae, and members of the family Hyphomicrobiaceae. Examples of the class Betaproteobacteria include members of the order Burkholderiales, and examples of members of the order Burkholderiales include members of the family Alcaligenaceae, members of the family Burkholderiaceae, and members of the family Sutterellaceae. Examples of the class Deltaproteobacteria include members of the order Desulfovibrionales, and examples of members of this order include members of the family Desulfovibrionaceae and Desulfomicrobiaceae. Examples of the class Epsilonproteobacteria include members of the order Desulfobacterales, and examples of members of this order include members of the family Desulfobacteraceae. Examples of the class Gammaproteobacteria includes members of the order Alteromonadales and Enterobacteriales. Examples of members of the order Alteromonadales include members of the family Shewanellaceae, and examples of members of the order Enterobacteriales include members of the family Enterobacteriaceae.

Other examples of prokaryotic cells include members of the phylum Tenericutes include members of the class Mollicutes. Examples of the class Mollicutes include members of the order Entomoplasmatales, and members of the order Entomoplasmatales include members of the family Spiroplasmataceae.

Other examples of prokaryotic cells include members of the class Verrucomicrobiae include members of the order Verrucomicrobiales, and examples of members of the order Verrucomicrobiales includes members of the family Verrucomicrobiaceae. Other examples of prokaryotic cells include members of the family Fusobacteriaceae.

In one embodiment a freeze-dried composition may include prokaryotic bacteria that are members of at least 1 phylum, at least 2 phyla, at least 3 phyla, at least 4 phyla, at least 5 phyla, at least 6 phyla, at least 7 phyla, at least 8 phyla, at least 9 phyla, or at least 10 phyla. In one embodiment a composition of the present invention may include prokaryotic bacteria that are members of at least 1 class, at least 2 classes, at least 3 classes, at least 4 classes, at least 5 classes, at least 6 classes, or at least 7 classes. In one embodiment a composition of the present invention may include prokaryotic bacteria that are members of at least 1 order, at least 2 orders, at least 3 orders, at least 4 orders, at least 5 orders, at least 6 orders, or at least 7 orders. In one embodiment a composition of the present invention may include prokaryotic bacteria that are members of at least 1 family, at least 2 families, at least 3 families, at least 4 families, at least 5 families, at least 6 families, at least 7 families. In one embodiment a composition of the present invention may include at least 5, at least 10, at least 20, or at least 30 different genera of prokaryotic bacteria. In one embodiment a composition of the present invention may include at least 10, at least 50, at least 100, at least 200, at least 300, at least 400, at least 500, at least 600, or at least 700 different species of prokaryotic bacteria.

In one embodiment a freeze-dried composition described herein includes, when reconstituted with water, no greater than 10% weight of non-living material/weight biological material (wt/wt), no greater than 5% (wt/wt), no greater than 2.5% (wt/wt), no greater than 1% (wt/wt), no greater than 0.1% (wt/wt), no greater than 0.01% (wt/wt), or no greater than 0.001% (wt/wt) non-living material. In one embodiment, the amount of non-living material in a composition of the present invention is undetectable using currently available techniques. For instance, living material can be stained for biological activity, electron transport, DNA and RNA for specific genes.

In one embodiment, the fecal material present in a freeze-dried composition described herein does not include, when reconstituted with water, particles (e.g., particles of non-living material and/or particles of biological material) having a size of greater than 2.0 millimeters (mm), greater than 1.0 mm, greater than 0.5 mm, greater than 0.4 mm, greater than 0.3 mm, greater than 0.25 mm, greater than 0.212 mm, greater than 0.180 mm, greater than 0.150 mm, greater than 0.125 mm, greater than 0.106 mm, greater than 0.090 mm, greater than 0.075 mm, greater than 0.063 mm, greater than 0.053 mm, greater than 0.045 mm, greater than 0.038 mm, greater than 0.032 mm, greater than 0.025 mm, greater than 0.020 mm, or greater than 0.01 mm. Non-fecal material present in a composition may include particles having a size of greater than 2.0 mm, greater than 1.0 mm, greater than 0.5 mm, greater than 0.4 mm, greater than 0.3 mm, greater than 0.25 mm, greater than 0.212 mm, greater than 0.180 mm, greater than 0.150 mm, greater than 0.125 mm, greater than 0.106 mm, greater than 0.090 mm, greater than 0.075 mm, greater than 0.063 mm, greater than 0.053 mm, greater than 0.045 mm, greater than 0.038 mm, greater than 0.032 mm, greater than 0.025 mm, greater than 0.020 mm, or greater than 0.01 mm. In one embodiment, the fecal material present in a composition of the present invention consists of, or consists essentially of, particles of non-living material and/or biological material having a size that will pass through a sieve having a sieve size of 2.0 mm, 1.0 mm, 0.5 mm, 0.4 mm, 0.3 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, or 0.01 mm. Thus, in such an embodiment, the fecal material present in a composition has a size that is less than or equal to 2.0 mm, less than or equal to 1.0 mm, less than or equal to 0.5 mm, less than or equal to 0.4 mm, less than or equal to 0.3 mm, less than or equal to 0.25 mm, less than or equal to 0.212 mm, less than or equal to 0.180 mm, less than or equal to 0.150 mm, less than or equal to 0.125 mm, less than or equal to 0.106 mm, less than or equal to 0.090 mm, less than or equal to 0.075 mm, less than or equal to 0.063 mm, less than or equal to 0.053 mm, less than or equal to 0.045 mm, less than or equal to 0.038 mm, less than or equal to 0.032 mm, less than or equal to 0.025 mm, less than or equal to 0.020 mm, or less than or equal to 0.01 mm. The sieve size may be based on the US Standard sieve sizes of, for instance, 10, 18, 35, 60, 70, 80, 100, 120, 140, 170, 200, 230, 270, 325, or 400.

A composition of the present invention may optionally include a cryoprotectant. A cryoprotectant is a compound that maintains the viability of fecal microbes when frozen. Cryoprotectants are known in the art and used routinely to protect microbes when exposed to freezing conditions. Examples include, but are not limited to, amino acids such as alanine, glycine, proline; simple sugars such as sucrose, glucose, lactose, ribose, and trehalose; and other compounds such as dimethyl sulfoxide (DMSO), and glycerol. The amount of cryoprotectant present in a composition described herein may vary depending on the cryoprotectant used and the temperature to be used for freezing (e.g., −20° C., −80° C., or a different temperature). The amount of cryoprotectant that can be used is known to the skilled person or may be easily determined using routine experimentation. In one embodiment, a composition of the present invention may include glycerol at a concentration of 10%.

A freeze-dried composition described herein includes a cryoprotectant. Cryoprotectants useful in freeze-drying microbes are known and include, for instance, D-Mannitol, D-Sorbitol, D-Glucose, casein hydrolysate, sucrose, gelatin, non-fat skim milk, starch hydolysate, fetal calf serum, bovine serum albumin, or combinations of 1, 2, 3, or 4 of the above cryoprotectants. Other cryoprotectants are also known. A cryoprotectant useful herein maintains the viability of fecal microbes when subjected to freeze-drying conditions, milling or grinding, and/or when stored as a freeze-dried composition. Milling, also referred to as grinding, is a process that physically changes a material into smaller particles. Methods for milling freeze-dried compositions are known to the skilled person, and can occur at various temperatures, e.g., at or below 0° C., or above 0° C. A cryoprotectant useful herein results in a freeze-dried composition that is friable. As used herein, a “friable” composition refers to a composition that can be easily milled to result in a fine powder. In one embodiment, a freeze-dried composition described herein that is friable is one that results in a powder that can be subsequently used to produce a tablet. In one embodiment, a useful powder may have size, density, flow, and compression characteristics suitable for production of tablets or encapsulation.

Useful cryoprotectants include those that result in a composition that is friable, does not crystallize during freeze-drying, and maximizes survival of microbes. In one embodiment, examples of useful cryoprotectants include, but are not limited to, skim milk, gelatin, and mannitol, and sucrose. In one embodiment, more than one cryoprotectant may be used, such as the combination of sucrose (5%) and skim milk (5%), or the combination of sucrose (10%) and gelatin (0.1%).

In one embodiment, a useful cryoprotectant is not sucrose alone, which unexpectedly crystallizes and hardens during freeze-drying, or glycerol, which unexpectedly results in an oily and viscous composition upon freeze-drying with a fecal material as described herein.

The total cryoprotectant used to produce a freeze-dried composition may be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% (vol/vol) of the final concentration of a mixture of fecal microbes and the cryoprotectant before freeze-drying the composition. For instance, to produce a composition having a cryoprotectant at a final concentration of 10%, equal volumes of a 20% solution of the cryoprotectant and a mixture of microbes derived from fecal material can be combined and mixed, and then freeze-dried.

In one embodiment a freeze-dried composition of the present invention does not include pathogenic biological material. In one embodiment, fecal material is from a person that has undergone a medical history, a physical examination, and laboratory testing. The evaluation of medical history may include, but is not limited to, risk of infectious agents, presence of gastrointestinal co-morbidities, factors that can or do affect the composition of the intestinal microbiota, and systemic medical conditions. Exclusion criteria regarding risk of infectious agents may include, but are not limited to, known viral infection with Hepatitis B, C or HIV; known exposure to HIV or viral hepatitis at any time; high risk behaviors including sex for drugs or money, men who have sex with men, more than one sexual partner in the preceding 12 months, any past use of intravenous drugs or intranasal cocaine, history of incarceration; tattoo or body piercing within 12 months; travel to areas of the world where risk of traveler's diarrhea is higher than the US; and current communicable disease, e.g., upper respiratory viral infection.

Exclusion criteria regarding gastrointestinal co-morbidities include, but are not limited to, history of irritable bowel syndrome, wherein specific symptoms may include frequent abdominal cramps, excessive gas, bloating, abdominal distension, fecal urgency, diarrhea, constipation; history of inflammatory bowel disease such as Crohn's disease, ulcerative colitis, microscopic colitis; chronic diarrhea; chronic constipation or use of laxatives; history of gastrointestinal malignancy or known colon polyposis; history of any abdominal surgery, e.g., gastric bypass, intestinal resection, appendectomy, cholecystectomy, and the like; use of probiotics or any other over the counter aids used by the potential donor for purpose of regulating digestion, but yogurt and kefir products may be allowed if taken merely as food rather than nutritional supplements.

Exclusion criteria regarding factors that can or do affect the composition of the intestinal microbiota include, but are not limited to, antibiotics for any indication within the preceding 6 months; any prescribed immunosuppressive or anti-neoplastic medications.

Exclusion criteria regarding systemic medical conditions include, but are not limited to, established or emerging metabolic syndrome, where criteria used for definition here are stricter than established criteria, including history of increased blood pressure, history of diabetes or glucose intolerance; known systemic autoimmunity, e.g., connective tissue disease, multiple sclerosis; known atopic diseases including asthma or eczema; chronic pain syndromes including fibromyalgia, chronic fatigue syndrome; ongoing (even if intermittent) use of any prescribed medications, including inhalers or topical creams and ointments; neurologic, neurodevelopmental, and neurodegenerative disorders including autism, Parkinson's disease.

Exclusion criteria on physical examination may include, but are not limited to, general, such as body mass index<30 kg/m², central obesity defined by waste:hip ratio>0.90 (male) and >0.85 (female); blood pressure>135 mmHg systolic and >85 mmHg diastolic; skin—presence of a rash, tattoos or body piercing placed within a year, jaundice; enlarged lymph nodes; wheezing on auscultation; hepatomegaly or stigmata of liver disease; swollen or tender joints; muscle weakness; abnormal neurologic examination.

Exclusion criteria on laboratory testing may include, but is not limited to, positive stool Clostridium difficile toxin B tested by PCR; positive stool cultures for any of the routine pathogens including Salmonella, Shigella, Yersinia, Campylobacter, E. coli O157:H7; abnormal ova and parasites examination; positive Giardia, Cryptosporidium, or Helicobacter pylori antigens; positive screening for any viral illnesses, including HIV 1 and 2, Viral Hepatitis A IgM, Hepatitis surface antigen and core Ab; abnormal RPR (screen for syphilis); any abnormal liver function tests including alkaline phosphatase, aspartate aminotransaminase, alanine aminotransferase; raised serum triglycerides>150 mg/dL; HDL cholesterol<40 mg/dL (males) and <50 mg/dL (females); high sensitivity CRP>2.4 mg/L; raised fasting plasma glucose (>100 mg/dL).

The freeze-dried compositions of the present invention may be included in a diversity of pharmaceutically acceptable formulations. In one embodiment, a formulation may be a fluid composition. Fluid compositions include, but are not limited to, solutions, suspensions, dispersions, and the like. In one embodiment, a formulation may be a solid composition. Solid compositions include, but are not limited to, powder, granule, compressed tablet, pill, capsule, chewing gum, microsphere, wafer, and the like. Those formulations may include a pharmaceutically acceptable carrier to render the composition appropriate for administration to a subject. As used herein “pharmaceutically acceptable carrier” includes pharmacologically inactive compounds compatible with pharmaceutical administration. The compositions of the present invention may be formulated to be compatible with its intended route of administration. A composition of the present invention may be administered by any method suitable for depositing in the gastrointestinal tract, preferably the colon, of a subject. Examples of routes of administration include rectal administration (e.g., by suppository, enema, upper endoscopy, upper push enteroscopy, or colonoscopy), intubation through the nose or the mouth (e.g., by nasogastric tube, nasoenteric tube, or nasal jejunal tube), or oral administration (e.g., by a solid such as a pill, tablet, or capsule, or by liquid). In embodiments where a liquid form of the composition is delivered to a subject, the freeze-dried composition is reconstituted with an aqueous solution, such as by adding water or saline, or exposing the freeze-dried composition to a body fluid.

For therapeutic use in the method of the present invention, a composition described herein may be conveniently administered in a form containing one or more pharmaceutically acceptable carriers. Suitable carriers are well known in the art and vary with the desired form and mode of administration of the composition. For example, they may include diluents or excipients such as fillers, binders, wetting agents, disintegrators, surface-active agents, glidants, lubricants, and the like. Typically, the carrier may be a solid (including powder), liquid, or combinations thereof. Each carrier is preferably “acceptable” in the sense of being compatible with the other ingredients in the composition and not injurious to the subject. The carrier is preferably biologically acceptable and inert, i.e., it permits the composition to maintain viability of the biological material until delivered to the appropriate site.

Oral compositions may include an inert diluent or an edible carrier. For the purpose of oral therapeutic administration, the freeze-dried composition can be incorporated with excipients and used in the form of tablets, or capsules, e.g., gelatin capsules. Oral compositions can also be prepared by combining a composition of the present invention with a food. In one embodiment a food used for administration is chilled, for instance, ice cream or milk. Pharmaceutically compatible binding agents, and/or adjuvant materials can be included as part of the composition. The tablets, pills, capsules, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Other ingredients may be added to a formulation to provide desired characteristics such as flow, compression, hardness, and taste.

The freeze-dried composition can also be prepared in the form of suppositories (e.g., with conventional suppository bases such as cocoa butter and other glycerides) or retention enemas for rectal delivery.

The freeze-dried composition may be prepared with carriers that will protect the microbes against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from, for instance, Alza Corporation and Nova Pharmaceuticals, Inc. Liposomal suspensions can also be used as pharmaceutically acceptable carriers. These can be prepared according to methods known to those skilled in the art.

In one embodiment, the freeze-dried composition may be present in a formulation that permits passage to the small intestine or colon. For instance, when the composition is to be administered orally, the dosage form may be formulated so the composition is not exposed to conditions prevalent in the gastrointestinal tract before the small intestine or colon, e.g., high acidity and digestive enzymes present in the stomach and/or intestine. In one embodiment, the dosage form may be formulated so the composition passes through the stomach and is released in conditions that include a pH of greater than 5.5, greater than 6, greater than 6.5, or greater than 7. In one embodiment, a freeze-dried composition may be prepared with an enteric coating. In one embodiment an enteric coating is acid-resistant to protect the composition from the low pH of the stomach and break down when exposed to a pH greater than present in the stomach. The encapsulation of compositions in an enteric coating for therapeutic use is routine in the art. Materials used for enteric coatings include fatty acids, waxes, shellac, plastics, and plant fibers. Examples include, but are not limited to, methyl acrylate-methacrylic acid copolymers, cellulose acetate succinate, hydroxy propyl methyl cellulose phthalate, enteric coatings (hydroxypropyl methylcellulose (HPMC), hydroxy propyl methyl cellulose acetate succinate (hypromellose acetate succinate), polyvinyl acetate phthalate (PVAP), methyl methacrylate-methacrylic acid copolymers, cellulose acetate trimellitate, and sodium alginate. Encapsulation may include hard-shelled capsules, which may be used for dry, powdered ingredients, or soft-shelled capsules. Capsules may be made from aqueous solutions of gelling agents such as animal protein (e.g., gelatin), plant polysaccharides or derivatives like carrageenans and modified forms of starch and cellulose. Other ingredients may be added to a gelling agent solution such as plasticizers (e.g., glycerin and or sorbitol), coloring agents, preservatives, disintegrants, lubricants, and surface treatment. Enteric coated capsules can be co-combined to provide for release of the freeze dried composition within the large bowel or colon.

Useful diluents include aqueous solutions that are routinely used for manipulating microbes, eukaryotic cells, and/or viruses. Useful diluents may include constituents to maintain physiological buffer, osmolarity, and the like. The diluent is preferably sterile and/or non-allergenic. An example of a diluent includes, but is not limited to, phosphate buffered saline at pH 7. In one embodiment, 1 part donor feces may be combined with 5 parts diluent (e.g., 50 grams of donor feces may be combined with 250 mls diluent) and blended. In one embodiment, the oxygen in the blending chamber may be decreased or removed by purging with an inert gas such as nitrogen or argon prior to blending. Such anaerobic conditions may be useful to maintain viability of most anaerobic bacteria present in a colon. The sample may be blended multiple times and/or more diluent may be added until a consistency is achieved that will permit the following steps to occur. In one embodiment, anaerobic conditions are not used in steps following the blending. It was found that anaerobic conditions were not necessary in the steps following the blending, and this was unexpected and surprising since a substantial percentage of prokaryotic cells in fecal material are strict anaerobes, and exposure to oxygen kills them. After the blending, the solutions used for washing and resuspension did not need to be purged of oxygen, and manipulation of the microbiota in an oxygen-free cabinet or glove box was not needed.

In one embodiment, a composition may be prepared by obtaining a fecal sample from an appropriate donor and blending with a diluent as described in Sadowsky et al. (WO 12/122478). In another embodiment, a composition may be prepared by obtaining a fecal sample from an appropriate donor and using ballistic disruption with a horizontal or vertical shaker, a suitable diluent and stainless steel beads of 3.2 mm in diameter. The mixture is shaken to break up the sample and the beads are removed from the suspension by filtration a stainless steel strainer. The suspension is centrifuged at a suitable speed to pellet the microbes, for instance, 500-1,000 rpm, the supernatant poured off and the resulting microbial fraction obtained by selective filtration.

Not all microbes and eukaryotic cells present in an individual's colon can be cultured, thus, in one embodiment conditions for preparing a freeze-dried composition include the use of temperatures that decrease the replication of the microbes and eukaryotic cells. In one embodiment, the conditions used for preparation are maintained below 37° C. For instance, the conditions used for preparation are maintained at a temperature of no greater than 30° C., no greater than 20° C., no greater than 10° C., or no greater than 5° C. In one embodiment, conditions are used such that replication of the microbes and eukaryotic cells is undetectable, and preferably does not occur. When the conditions used to prepare a composition of the present invention include lower temperatures to minimize replication and cell death, the biological material present in a composition includes a population of microbes, eukaryotic cells, and viruses that is essentially identical to a population of microbes, eukaryotic cells, and viruses present in the colon or feces of a normal healthy human, e.g., the donor from whom the fecal sample was obtained. In one embodiment, the conditions used for preparation decrease exposure of the microbes and eukaryotic cells to oxygen, both before and after purification of microbiota.

Removal of non-living material may be achieved by passing the blended sample through a sieve with a sieve size of no greater than 2.0 mm, no greater than 1.0 mm, no greater than 0.5 mm, no greater than 0.25 mm, no greater than 0.212 mm, no greater than 0.180 mm, no greater than 0.150 mm, no greater than 0.125 mm, no greater than 0.106 mm, no greater than 0.090 mm, no greater than 0.075 mm, no greater than 0.063 mm, no greater than 0.053 mm, no greater than 0.045 mm, no greater than 0.038 mm, no greater than 0.032 mm, no greater than 0.025 mm, no greater than 0.020 mm, no greater than 0.01 mm, or no greater than 0.2 mm. In one embodiment, the blended sample is prepared by passing it through a sieve with a sieve size of 0.25 mm and collecting the filtrate. In one embodiment, the blended sample is passed through sieves with progressively smaller sieve sizes until final passage through a sieve size of 0.25 mm. For instance, if a total of four sieves are used the sieve size of the first sieve may be 2 mm, followed by 1 mm, followed by 0.5 mm, and then followed by 0.25 mm. The final filtrate may be collected in a centrifuge tube, and centrifuged at a speed sufficient to pellet the biological material, for instance, 10,000×g for 10 minutes at 4° C. The supernatant is removed, the cells are resuspended in diluent, optionally centrifuged again, for instance at 10,000×g for 10 minutes at 4° C. The final supernatant is discarded and the cells are resuspended in an aqueous solution (e.g., diluent, cryoprotectant, and the like, or a combination thereof). In one embodiment, the volume of the blended mixture is decreased through the steps of sieving and washing. For instance, in one embodiment, the volume is decreased to 14% of the volume used in the blending (e.g., from 250 mls to 35 mls). In one embodiment, the volume of the blended mixture is decreased through the steps of sieving and washing to result in between 1×10¹⁰ and 5×10¹⁰ cells in a volume that is subsequently administered to a subject. The final filtrate may also be collected in a centrifuge tube, washed, and the cells resuspended in an aqueous solution (e.g., diluent, cryoprotectant, and the like, or a combination thereof). In one embodiment, the volume of the blended mixture is decreased through the steps of sieving and washing to result in at least 1×10¹² cells, for instance, at least 1.5×10¹² cells, at least 2×10¹² cells, or at least 2.5×10¹² cells in a volume that is subsequently freeze-dried. Since most biological material is difficult or impossible to culture, a hemocytometer may be used to determine the number of cells. This process results in an extract of feces that is highly enriched for all colon microbiota that are able to pass through a sieve as described above, and can be centrifuged at 10,000×g for 10 minutes. As used herein, “enriched” refers to increasing the abundance of biological material relative to non-living material, such that biological material constitutes a significantly higher proportion compared to the fecal material before the enrichment. The term “enriched” refers to those situations in which a person has intervened to elevate the proportion of biological material.

The amount of aqueous solution added may be in an amount to result in a single dosage having an appropriate number of cells. In one embodiment, a single dosage may include between 1×10¹⁰ and 5×10¹⁰ cells, for instance, 3×10¹⁰ cells. Since most biological material is difficult or impossible to culture, a hemocytometer may be used to determine the number of cells.

In one embodiment the resulting pellet may be suspended in half the original volume of diluent containing 10% glycerol. The sample may be used immediately, or may be frozen, for instance, at −80° C., for later use. When freezing, the sample may be left in a centrifuge tube, or may be in a different container. In one embodiment, the container is one that increases the surface area of the sample. For instance, the sample may be placed in an IV bag. When the frozen sample is to be used, it may be thawed on ice and then transplanted into the recipient. It was found that freezing the compositions described herein did not result in destruction of its curative potential. In one embodiment the sample resulting from centrifugation may be processed for long term storage of 1 year or longer. The ability to store such a sample provides a level of flexibility that was not possible with other methods. For instance, it was necessary to quickly identify a donor, rapidly process a fecal sample from the donor, and use it immediately. Examples of useful processing methods include, but are not limited to, freezing, freeze drying, spray drying, lyophilization, vacuum drying, air drying, or other forms of evaporative drying. Processing of a composition of the present invention may include the production of a powder following any drying procedure.

The use of sieves to extract biological material from fecal material unexpectedly resulted in a composition which was nearly odorless. This was not expected because feces normally have a distinctive odor and this was surprising to be removed by the minimal manipulation used. This is a significant advantage as it takes a method that is unaesthetic and so distasteful that some patients and staff refuse to take part, and changes it into a method that is easily practiced in a normal clinical setting or at home. As used herein, “odorless” means there is a decreased amount of volatile organic molecules present, and the decreased amount of volatile organic molecules present can be easily detected by a person comparing the material before processing with the material after processing.

In one embodiment, a composition described herein of fecal microbes is freeze-dried to form solid dried powder. A composition of fecal microbes is mixed with a cryoprotectant and subjected to conditions that result in freeze-drying. Such conditions typically include freezing the sample, and reducing the pressure surrounding the frozen sample to remove water from the sample. Once freeze-dried, the composition may be further processed by subjecting the dried material to force sufficient to break up the material into a powder that can be easily stored until used. In one embodiment, the powder may be used to form granules, compressed tablets, pills, capsules, wafers, and the like. In one embodiment, the freeze dried material can be formulated such that it is released from a capsule into the small or large intestine or the colon, and not the stomach.

The present invention is further directed to methods of using the freeze-dried compositions described herein. One method includes administering to a subject in need thereof an effective amount of a composition described herein. The administering is under conditions suitable for deposition of the composition in a region of the large or small intestine such that the biological material in the composition colonizes the colon. For instance, administration may be into upper gastrointestinal tract, as well as lower gastrointestinal tract, e.g., the terminal ileum, cecum, colonic areas containing diverticulosis, and rectum. In one embodiment the administering may be oral, such as by tablet. In one embodiment the administering may be by intubation, such as by nasogastric tube, of a freeze-dried composition that has been reconstituted. In one embodiment the administering may be rectal, for instance by a colonoscope, enema, or suppository. Conditions that are “suitable” for an event to occur, or “suitable” conditions are conditions that do not prevent such events from occurring. Thus, these conditions permit, enhance, facilitate, and/or are conducive to the event. As used herein, an “effective amount” relates to a sufficient amount of a composition described herein, to provide the desired effect. For instance, in one embodiment an “effective amount” is an amount effective to alleviate one or more symptoms and/or signs of the disease as described herein. In some embodiments, an effective amount is an amount that is sufficient to effect a reduction in a symptom and/or sign associated with a disease, such as diarrhea or C. difficile. A reduction in a symptom and/or a sign is, for instance, at least 10%, at least 20%, at least 30%, at least 40%, at least 50%, at least 60%, at least 70%, at least 80%, at least 90%, or at least 100% in a measured sign as compared to a control, a non-treated subject, or the subject prior to administration of the composition. In one embodiment, an effective amount is an amount sufficient to result in at least 1×10¹², at least 1.5×10¹², at least 2×10¹², or at least 2.5×10¹² cells administered to the subject. In one embodiment, an effective amount is an amount sufficient to result in at least 1×10¹², at least 1.5×10¹², at least 2×10¹², or at least 2.5×10¹² cells delivered to the colon. In one embodiment, an effective amount is an amount sufficient to result in 1×10¹² to 3×10¹² cells delivered to the colon, or 1.5×10¹² to 2.5×10¹² cells delivered to the colon. It will be understood, however, that the total dosage of the compositions as disclosed herein will be decided by the attending physician within the scope of sound medical judgment. The exact amount required will vary depending on factors such as the type and extent of disease being treated.

In one embodiment, a method of the present invention includes treating certain diseases in a subject in need of treatment. The subject may be a mammal, such as a human. In some embodiments animal models may be used, such as a mammal, including a rat, a mouse, a hamster, a gerbil, or a primate. As used herein, the term “disease” refers to any deviation from or interruption of the normal structure or function of a part, organ, or system, or combination thereof, of a subject that is manifested by a characteristic symptom or clinical sign. Diseases include those characterized by dysfunctional composition of colon microbiota. Such diseases include, but are not limited to, colitis, including autoimmune colitis (e.g., inflammatory bowel disease, ulcerative colitis, Crohn's disease, irritable bowel syndrome) and infectious colitis. Examples of infectious colitis include, but are not limited to Clostridium difficile colitis (e.g., acute C. difficile colitis, relapsing C. difficile colitis, or severe C. difficile colitis) and enterohemorrhagic colitis (e.g., a colitis caused by Shigella spp. or E. coli). Other examples of diseases include, but are not limited to, chronic diarrhea; chronic constipation, metabolic syndrome and obesity, atopic diseases including asthma, eczema, eosinophilic disorders of the GI tract, systemic autoimmunity including rheumatoid arthritis, systemic lupus erythematosis, multiple sclerosis, etc., chronic pain disorders such fibromyalgia, chronic fatigue syndrome, neurodegenerative disorders, eating disorders, and malnutrition.

As used herein, the term “symptom” refers to subjective evidence of disease or condition experienced by the patient and caused by disease. As used herein, the term “clinical sign,” or simply “sign,” refers to objective evidence of a disease present in a subject. Symptoms and/or signs associated with diseases referred to herein and the evaluation of such signs are routine and known in the art. Typically, whether a subject has a disease, and whether a subject is responding to treatment, may be determined by evaluation of signs associated with the disease.

Treatment of a disease can be prophylactic or, alternatively, can be initiated after the development of a disease. Treatment that is prophylactic, for instance, initiated before a subject manifests signs of a disease, is referred to herein as treatment of a subject that is “at risk” of developing a disease. An example of a subject that is at risk of developing a disease is a person having a risk factor. An example of a risk factor for Clostridium difficile colitis is antibiotic therapy of the gastrointestinal tract. Treatment can be performed before, during, or after the occurrence of the diseases described herein. Treatment initiated after the development of a disease may result in decreasing the severity of the signs of the disease, or completely removing the signs.

In one embodiment, a method of the present invention includes transplanting a microbiota from a donor to a recipient.

In one embodiment, a method of the present invention includes increasing the relative abundance of members of the phylum Firmicutes, such as a non-pathogenic member of the class Clostridia, and/or members of the phylum Bacteroidetes, in a recipient's colon. The phrase “relative abundance” refers to number of members of a phylum or class compared to the number of members of all other taxa in a recipient's colon. Such a comparison can be expressed as a percent. In one embodiment, the relative abundance of non-pathogenic members of the class Clostridia in a recipient's colon after the administration may be increased by at least 5%, at least 10%, at least 20%, or at least 50%, compared to the recipient's colon before the administration. In one embodiment, the relative abundance of members of the phylum Firmicutes in a recipient's colon after the administration may be increased by at least 5%, at least 10%, at least 20%, or at least 50% compared to the recipient's colon before the administration. The change in the abundance may be determined at, for instance, 3 days, 10 days, 15 days, or 25 days after the administration of fecal microbiota.

In one embodiment, a method of the present invention includes decreasing the relative abundance of members of the phylum Proteobacteria in a recipient's colon. In one embodiment, the relative abundance of members of the phylum Proteobacteria in a recipient's colon after the administration may be decreased by at least 10%, at least 20%, at least 30%, or at least 40% compared to the recipient's colon before the administration. The change in the abundance of members of the phylum Proteobacteria may be determined at, for instance, 3 days, 10 days, 15 days, or 25 days after the administration.

In one embodiment, the existing microbiota does not need to be cleared prior to administration of a freeze-dried composition of the present invention. In other embodiments clearance of the microbiota may be necessary. Methods for clearance of existing microbiota are known and routine. In one example, clearance can be accomplished by administering a cocktail of antibiotics for one week until a day prior to transplant. An example of a useful cocktail is Metronidazole (1000 mg twice daily), Rifaximin (550 mg twice daily), Vancomycin (500 mg twice daily), and Neomycin (1000 mg twice daily).

The present application further includes the following exemplary embodiments.

Embodiment 1: A composition comprising an extract or preparation of human feces wherein the composition comprises no greater than about 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% weight non-living material/weight biological material, wherein optionally the biological material comprises human gut, colon or intestinal fecal microbes, and optionally the biological material comprises human gut, colon or intestinal bacteria, wherein optionally the composition comprises a pharmaceutically acceptable carrier, and optionally the composition is a formulation for oral administration.

Embodiment 2: A composition comprising an extract or preparation of human feces comprising human fecal material, wherein the human fecal material consists of, or consists essentially of, particles of non-living material and/or particles of biological material that will pass through a sieve having a sieve size of 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.2 mm, wherein optionally the composition comprises a pharmaceutically acceptable carrier, and optionally the composition is a formulation for oral administration.

Embodiment 3: A composition comprising at least 4 different phyla of gut, colon or intestinal bacteria extracted or prepared from the gut, colon or intestine, wherein the phyla comprise a Bacteroidetes, a Firmicutes, a Proteobacteria a Tenericutes phylum, or a combination thereof, wherein optionally the phyla are chosen from Bacteroidetes, Firmicutes, Proteobacteria, Tenericutes, or a combination thereof, and wherein the composition comprises no greater than about 0.05%, 0.1%, 0.2%, 0.3%, 0.4%, 0.5%, 0.6%, 0.7%, 0.8%, 0.9° A 1%, 2%, 3%, 4%, 5%, 6%, 7%, 8%, 9% or 10% weight non-living material/weight biological material, wherein optionally the biological material comprises human gut, colon or intestinal flora, and optionally the biological material comprises human gut, colon or intestinal bacteria, wherein optionally the composition comprises a pharmaceutically acceptable carrier, and optionally the composition is a formulation for oral administration.

Embodiment 4: A composition comprising an extract of human feces wherein the composition is substantially odorless, wherein the optionally the composition comprises biological material, and optionally wherein the biological material comprises bacteria, wherein optionally the composition comprises a pharmaceutically acceptable carrier, and optionally the composition is a formulation for oral administration.

Embodiment 5: The composition of any one of Embodiments 1 to 4, wherein the composition comprises no greater than about 0.1% weight non-living material/weight biological material.

Embodiment 6: The composition of Embodiment 2 wherein the fecal material consists of, or consists essentially of, particles that will pass through a 0.25 mm sieve, or equivalent.

Embodiment 7: The composition of Embodiment 2 wherein the composition comprises no greater than about 0.05%, 0.1%, 0.2%, 0.3%, 0.4%, 0.5%, 0.6%, 0.7%, 0.8%, 0.9%, 1%, 2%, 3%, 4% 5%, 6%, 7%, 8%, 9% or 10% weight non-living material/weight biological material.

Embodiment 8: The composition of Embodiment 1, 2, 3, or 4 wherein the composition further comprises a cryoprotectant.

Embodiment 9: The composition of Embodiment 8 wherein the cryoprotectant comprises a glycerol.

Embodiment 10: The composition of Embodiment 8 wherein the composition is at a temperature of less than about 0° C.

Embodiment 11: The composition of Embodiment 1, 2, 3 or 4 wherein the composition is a solid.

Embodiment 12: The composition of Embodiment 11 wherein the solid comprises a powder.

Embodiment 13: The composition of Embodiment 1, 2, 3 or 4 wherein the composition comprises at least about 1×10¹⁰, 2×10¹⁰, 3×10¹⁰, 4×10¹⁰, or 5×10¹⁰ bacteria.

Embodiment 14: The composition of any of Embodiments 1 to 13, wherein the biological material comprises: a plurality of prokaryotic cells, eukaryotic cells, or viruses; or a population of prokaryotic cells, eukaryotic cells, and viruses, that is substantially identical to or representative of or equivalent to a population of prokaryotic cells, eukaryotic cells, and viruses present in a feces of a normal healthy human.

Embodiment 15: The composition of Embodiment any of Embodiments 1 to 13, wherein the biological material present comprises a population of prokaryotic cells and viruses that is substantially identical to or representative of or equivalent to a population of prokaryotic cells and viruses present in the feces of a normal healthy human.

Embodiment 16: The composition of Embodiment 2 or 4 wherein the composition comprises a biological material, and the biological material comprises a population of prokaryotic cells, eukaryotic cells, or viruses that is substantially identical to or representative of or equivalent to a population of prokaryotic cells, eukaryotic cells, and viruses present in the feces of a normal healthy human.

Embodiment 17: A composition prepared by a process comprising: subjecting a fecal sample to a condition or conditions that remove at least about 91%, 92%, 93%, 94% 95%, 96%, 97%, 98%, 99% or more of the non-living material present in the fecal sample prior to the subjecting, wherein the optionally the composition comprises a biological material, and optionally the biological material comprises bacteria, wherein optionally the composition comprises a pharmaceutically acceptable carrier, and optionally the composition is a formulation for oral administration.

Embodiment 18: The composition of Embodiment 17 wherein the subjecting occurs at a temperature of no greater than about 26° C., 27° C., 28° C., 29° C., 30° C., 31° C., 32° C., 33° C., or 34° C.

Embodiment 19: A composition prepared by a process comprising: filtering a fecal sample with a filter medium, wherein the filter medium comprises a sieve size of no greater than about 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.2 mm to result in or to generate a filtrate, wherein the optionally the composition comprises a biological material, and optionally the biological material comprises bacteria, wherein optionally the composition comprises a pharmaceutically acceptable carrier, and optionally the composition is a formulation for oral administration.

Embodiment 20: The composition of Embodiment 19 further comprising filtering the fecal sample with a filter medium, wherein the filter medium comprises a pore sieve size of no greater than 0.25 mm.

Embodiment 21: The composition of Embodiment 19 wherein the filtering occurs at a temperature of no greater than about 26° C., 27° C., 28° C., 29° C., 30° C., 31° C., 32° C., 33° C., or 34° C.

Embodiment 22: The composition of Embodiment 17 or 19 wherein the composition comprises at least 4 different phyla of bacteria, wherein the phyla comprise a Bacteroidetes, a Firmicutes, a Proteobacteria, a Tenericutes phyla, or a combination thereof, wherein optionally the phyla are chosen from Bacteroidetes, Firmicutes, Proteobacteria, Tenericutes, or a combination thereof.

Embodiment 23: The composition of Embodiment 22 wherein the composition further comprises at least 5, 6, 7, 8, 9, or 10 different classes of bacteria chosen from Actinobacteria, Bacteroidia, Bacilli, Clostridia, Erysipelotrichi, Alphaproteobacteria, Betaproteobacteria, Gammaproteobacteria, Mollicutes, and Verrucomicrobiae.

Embodiment 24: The composition of Embodiment 17 or 19 wherein the process further comprises adding a cryoprotectant to the composition.

Embodiment 25: The composition of Embodiment 24 wherein the process further comprises freezing the composition.

Embodiment 26: A composition as described in Embodiment 1, 2, 3, or 4 for use as a therapeutic agent.

Embodiment 27: A composition as described in Embodiment 1, 2, 3, or 4 for use in the treatment of a disease or a pathological or iatrogenic condition of the colon.

Embodiment 28: The composition of Embodiment 28 wherein the disease is a disease or condition characterized by a dysfunctional or pathological composition of colon microbiota.

Embodiment 29: The composition of Embodiment 29 wherein the disease is a Clostridium difficile colitis.

Embodiment 30: A method for replacing or supplementing or modifying a subject's colon microbiota, the method comprising: administering to the subject the composition of Embodiment 1, 2, 3, 4, 16, 17, or 19.

Embodiment 31: A method for treating a subject, the method comprising: administering to a subject in need thereof an effective amount of the composition of Embodiment 1, 2, 3, 4, 16, 17, or 19.

Embodiment 32: The method of Embodiment 30 or 31 further comprising removal of some, most, or substantially all of the subject's colon, gut or intestinal microbiota prior to the administering.

Embodiment 33: The method of Embodiment 31 wherein the subject has or is at risk for having a colitis.

Embodiment 34: The method of Embodiment 33 wherein the colitis is an autoimmune colitis.

Embodiment 35: The method of Embodiment 34 wherein the autoimmune colitis is selected from an inflammatory bowel disease, an ulcerative colitis, a Crohn's disease and an irritable bowel syndrome.

Embodiment 36: The method of Embodiment 33 wherein the colitis is an infectious colitis.

Embodiment 37: The method of Embodiment 36 wherein the infectious colitis is selected from a Clostridium difficile colitis and an enterohemorrhagic colitis.

Embodiment 38: The method of Embodiment 37, wherein the Clostridium difficile colitis is selected from an acute Clostridium difficile colitis, a relapsing Clostridium difficile colitis, and a severe Clostridium difficile colitis.

Embodiment 39: The method of Embodiment 37 wherein the enterohemorrhagic colitis is caused by a microbe selected from a Shigella spp. and an E. coli.

Embodiment 40: The method of Embodiment 31 wherein the subject has or is at risk for chronic diarrhea or chronic constipation.

Embodiment 41: Use of a composition of any of Embodiments 1 to 29, for the manufacture of a medicament.

Embodiment 42: Use of a composition of any of Embodiments 1 to 29, for the manufacture of a medicament for treating or ameliorating or preventing a disease or a pathological or iatrogenic condition of the colon, wherein optionally the disease is a disease or condition characterized by a dysfunctional or pathological composition of colon microbiota, or the disease is a Clostridium difficile colitis, or the disease or condition is a colitis, an autoimmune colitis, an infectious colitis or an enterohemorrhagic colitis.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Clostridium difficile associated disease is a major known complication of antibiotic therapy. The pathogen is normally held in check by native colon microbiota, but this level of protection may be lost when these microbial communities are suppressed by antibiotics. Antibiotics used to treat C. difficile infection may also perpetuate its recurrence by continued suppression of normal microbiota. Thus, a significant fraction of patients suffer from recalcitrant C. difficile infection, and recalcitrant C. difficile infection is associated with significant morbidity. Fecal bacteriotherapy is an increasingly used method used to break the cycle of C. difficile infection recurrence presumably through restoration of normal intestinal microbial communities. We previously reported, in one clinical case, that bacteriotherapy of colon microbiota resulted in the replacement of a host's microbiota by that of the donor (Khoruts, et al., 2010, J. Clin. Gastroenterol., 44(5):354). In order to obtain a greater understanding of the composition and stability of microbial communities before and after bacteriotherapy, we have analyzed amplified 16S rRNA regions of fecal DNA (V5 and V6) by using a pyrosequencing technology (an Illumina HiSeq2000 or other Illumina platforms). Additional individuals are currently being processed and analyzed.

### Introduction

Clostridium difficile is an emerging pathogen and the most common cause of nosocomial diarrhea.

Infections are often associated with antibiotic therapy, where the protective effect provided by the normal intestinal flora is disrupted.

C. difficile infection is often controlled by additional antimicrobial therapy, but approximately 20% of patients develop refractory disease resulting in recurrent diarrhea.

Bacteriotherapy, in the form of a fecal transplantation, has been shown to successfully treat refractory C. difficile infection.

Next generation sequencing technologies have allowed for a deeper interrogation of the intestinal microflora and was used in our study to examine changes in microbial community structure after transplantation.

Donor fecal material was obtained from the patient's son, who was tested for infectious disease, including C. difficile, Hepatitis A, B, or C viruses, HIV virus, Salmonella, Campylobacter, Yersinia, Shigella, E. Coli 0157:H7, Helicobacter pylori, Treponema pallidum, Giardia, and Cryptosporidium.

The patient was infused with donor fecal material by colonoscopy, which revealed severe, extensive diverticulosis in the sigmoid colon. The donor's fecal material was deposited into the cecum. Symptoms consistent with C. difficile infection were resolved within days of bacteriotherapy.

### Methods

Patient fecal samples were collected at day −31 before the fecal transplant bacteriotherapy and at days 5, 21, 46, 95, 132, 159, 188, and 227 post transplantation. A donor fecal sample was collected the day of the procedure and deposited into the recipient's cecum.

DNA was extracted from fecal materials using a MOBIO ultra-clean fecal DNA kit (MOBIO Laboratories, Inc., Carlsbad, Calif.) as directed by the manufacturer. Triplicate samples were extracted and pooled.

The V6 hypervariable region of the bacteria 16S rRNA gene was amplified using 50 ng of extracted DNA as template. Barcoded primers were used for multiplex sequencing (Kysela et al., 2005, Environmental Microbiology 7:356-64, and Claesson et al., Nucleic Acids Research, 2010, Vol. 38, No. 22 e200 doi:10.1093/nar/gkq873). Triplicate samples were prepared and pooled.

Amplified samples were mixed in equal molar ratios and sequenced at the National Center for Genomic Research (NCGR) using the Illumina sequencing platform.

Sequence data was analyzed using MOTHUR and the SILVA reference database (Scholss, 2009, Appl. Environ. Microbiol., 75(23):7537-7541. The taxonomy of operational taxonomic units (OTUs) were assigned at the 97% similarity using the GreenGenes reference files.

Principal component analysis was done using Yue and Clayton's Theta calculation (Yue and Clayton, 2005, Commun. Stat. Theor. Methods, 34:21232131). Accumulation curves were calculated based on 97% OTU similarities.

### Results & Discussion

Greater than 40% of the sequences obtained from the recipient's pretransplantation sample (day −31) belonged to unclassified Mollicutes strains or the Gammaproteobacteria.

In contrast, the donor's and recipient's post-transplantation samples were dominated by Firmicutes. Unclassified members of the Clostridiales and the Ruminococcaceae family were abundant.

Community analysis done using the Yue and Clayton's theta index showed that the post-transplantation samples clustered more closely with each other and with the donor sample, compared to that of the recipient's pre-transplantation sample.

Sequence analysis indicated that the taxa present in the recipient's pre- and post-transplant fecal samples differed considerably, suggesting that fecal bacteriotherapy was successful in altering the patient's intestinal microflora.

The transplanted microbial community in the recipient's intestine remained fairly stable after 7.5 months post transplantation.

Surprisingly, sequences representing the Bacteroidales were in fairly low abundance in all of the samples analyzed.

### Example 2

### Donor Screening for Fecal Microbiota Material Preparation

The donor undergoes a complete medical history and physical examination. In addition, a full-length donor history questionnaire is completed as recommended by the FDA for blood donors, and potential donors saying yes to any of the questions are excluded (http://wwvv.fda.gov/downloads/BiologicsBloodVaccines/BloodBloodProducts/ApprovedProducts/LicensedProductsBLAs/BloodDonorScreening/UCM213552.pdf). However, as gut microbiota have been associated or postulated to be involved with multiple medical conditions, the process of selection is more rigorous than that of the blood donors and includes virtually any systemic illness.

### Inclusion Criteria

1. Age>18
2. Ability to provide informed consent.

Exclusion Criteria

I. Medical History
A. Risk of infectious agent.
   1. Known viral infection with Hepatitis B, C or HIV
   2. Known exposure to HIV or viral hepatitis at any time
   3. High risk behaviors including sex for drugs or money, men who have sex with men, more than one sexual partner in the preceding 12 months, any past use of intravenous drugs or intranasal cocaine, history of incarceration.
   4. Tattoo or body piercing within 12 months.
   5. Travel to areas of the world where risk of traveler's diarrhea is higher than the US.
   6. Current communicable disease, e.g., upper respiratory viral infection.
B. Gastrointestinal comorbidities.
   1. History of irritable bowel syndrome. Specific symptoms may include frequent abdominal cramps, excessive gas, bloating, abdominal distension, fecal urgency, diarrhea, constipation.
   2. History of inflammatory bowel disease such as Crohn's disease, ulcerative colitits, microscopic colitis.
   3. Chronic diarrhea.
   4. Chronic constipation or use of laxatives.
   5. History of gastrointestinal malignancy or known colon polyposis.
   6. History of any abdominal surgery, e.g., gastric bypass, intestinal resection, appendectomy, cholecystectomy, etc.
   7. Use of Probiotics or any other over the counter aids used by the potential donor for purpose of regulating digestion. Yogurt and kefir products are allowed if taken merely as food rather than nutritional supplements.
C. Factors that can or do affect the composition of the intestinal microbiota.
   1. Antibiotics for any indication within the preceding 6 months.
   2. Any prescribed immunosuppressive or anti-neoplastic medications.
D. Systemic Medical Conditions.
   1. Metabolic Syndrome, established or emerging. Criteria used for definition here are stricter than any established criteria. These include history of increased blood pressure, history of diabetes or glucose intolerance.
   2. Known systemic autoimmunity, e.g., connective tissue disease, multiple sclerosis.
   3. Known atopic diseases including asthma or eczema.
   4. Chronic pain syndromes including fibromyalgia, chronic fatigue syndrome.
   5. Ongoing (even if intermittent) use of any prescribed medications, including inhalers or topical creams and ointments.
   6. Neurologic, neurodevelopmental, and neurodegenerative disorders including autism, Parkinson's disease.

II. Exclusion Criteria on Physical Examination.
1. General. Body mass index>26 kg/m², central obesity defined by waste:hip ratio>0.85 (male) and >0.80 (female).
2. Blood pressure>135 mmHg systolic and >85 mmHg diastolic.
3. Skin—presence of a rash, tattoos or body piercing placed within a year, jaundice.
4. Enlarged lymph nodes.
5. Wheezing on auscultation.
6. Hepatomegaly or stigmata of liver disease.
7. Swollen or tender joints. Muscle weakness.
8. Abnormal neurologic examination.

III. Exclusion Criteria on Laboratory Testing.
1. Positive stool Clostridium difficile toxin B tested by PCR.
2. Positive stool cultures for any of the routine pathogens including Salmonella, Shigella, Yersinia, Campylobacter, E. coli O157:H7.
3. Abnormal ova and parasites examination.
4. Positive Giardia, Cryptosporidium, or Helicobacter pylori antigens.
5. Positive screening for any viral illnesses, including HIV 1 and 2, Viral Hepatitis A IgM, Hepatitis surface antigen and core Ab.
6. Abnormal RPR (screen for syphilis).
7. Any abnormal liver function tests including alkaline phosphatase, aspartate aminotransaminase, alanine aminotransferase.
8. Raised serum triglycerides>150 mg/dL
9. HDL cholesterol<40 mg/dL (males) and <50 mg/dL (females)
10. High sensitivity CRP>2.4 mg/L
11. Raised fasting plasma glucose (>100 mg/dL)

### Example 3

### Fecal Sample Processing

Donor fecal material is immediately chilled on ice for transport to the laboratory. Samples are processed within one hour after collection.

Fecal samples are homogenized by mixing 50 g of donor feces and 250 ml of sterile phosphate buffered saline, pH 7, (PBS) in a Waring Blender. The blending chamber is purged with nitrogen gas for several minutes to remove oxygen prior to homogenization. Samples are blended three times on the lowest setting for 20 seconds. Additional PBS or blending cycles may be added depending on the consistency of the fecal suspension. Blended samples are passed through a series of four sieves with pore sizes of 2.0 mm, 1.0 mm, 0.5 mm and 0.25 mm (W.S. Tyler Industrial Group, Mentor, Ohio). The sieves were based on US standard sieve sizes of 10, 18, 35, and 60 for 2.0 mm, 1.0 mm, 0.5 mm and 0.25 mm, respectively. The final filtrate passing through the sieves (less than 0.25 mm fraction) is collected in 50 ml conical centrifuge tubes and centrifuged at 4,000 rpm (about 4,000×g) for 10 minutes at 4° C. The supernatant is discarded and the pellet is suspended in one half the original volume of PBS (e.g. 125 ml) containing 10% glycerol. The samples are used immediately, or stored frozen at −80° C. and thawed on ice before transplantation.

### Example 4

This example reports clinical experience with 43 consecutive patients that were treated for recurrent CDI C. difficile infection (CDI). During this time donor identification and screening was simplified by moving from patient-identified individual donors to standard volunteer donors. Material preparation shifted from the endoscopy suite to a standardized process in the laboratory, and ultimately to banking frozen processed fecal material that is ready to use when needed. Standardization of material preparation significantly simplified the practical aspects of treatment without loss of apparent efficacy in clearing recurrent CDI. Approximately 30% of the patients had underlying inflammatory bowel disease, and treat went was equally effective in this group. Several key steps in standardization of donor material preparation significantly simplified the clinical practice of treatment of recurrent CDI in patients failing antibiotic therapy. This is also reported in Hamilton et al., Am. J. Gastroenterol., 2012, doi:10.1038/ajg.2011.482.

### Methods

### Patients

This report includes the first 43 patients who received fecal microbiota transplantation (FMT) for recurrent CDI. All patients were identified by direct referral from clinicians at infectious disease and gastroenterology practices in the Minneapolis and St. Paul metropolitan area. Inclusion criteria for FMT included a history of symptomatic, toxin-positive, infection by C. difficile and at least two documented subsequent recurrences despite use of standard antibiotic therapy. At least one failed antibiotic regimen had to include a minimum of a 6 week course of tapered or pulsed vancomycin dosage, or at least a one month vancomycin course followed by a minimum two week rifaximin “chaser.” The only exclusion criteria in the protocol were age<18 and medical fragility from non-C. difficile problems resulting in life expectancy of <1 year. In the latter situation we advised patients that the best therapeutic option was an indefinite course of vancomycin. All patients gave informed consent for FMT via colonoscopy, recognizing relatively limited experience with this treatment approach and the intrinsic unknowns associated with its use. The Institutional Review Board at the University of Minnesota approved prospective collection of clinical outcome data, while recognizing this experience does not constitute a clinical trial, and as such was not designed to test the efficacy of FMT in comparison with any other therapeutic options.

### Donor Identification and Screening

At the start of the program patients were asked to self-identify potential donors. These included mothers (n=2), daughters (n=1), sons (n=3), wives (n=1), husbands (n=1), and friends (n=2). Prior to recruitment, the donors were required to submit available medical records and have a separate medical history interview away from the recipient patient. The history included: assessment of infectious risk, including identification of known risk factors for HIV and Hepatitis, current communicable diseases, and recent travel to areas of the world with a higher prevalence of diarrheal illnesses. Additional absolute donor exclusion criteria included gastrointestinal co-morbidities and the use of antibiotics within preceding three months. Since gut microbiota are likely involved in various aspects of energy metabolism and the functioning of the immune system, the presence of features of metabolic syndrome, autoimmunity, or allergic diseases were treated as relative exclusion criteria. Donors provided separate informed consent to participate in the protocol, which included risks associated with laboratory screening. The donors underwent serologic testing for HIV and Hepatitis B and C, and stool testing that included screening for routine enteric pathogens, C. difficile toxin B, and examination for ova and parasites, and Giardia and Cryptosporidium antigens.

Given varying logistic difficulties in recruiting individual patient-identified donors, the lack of availability of donor materials when needed, and no evidence to suggest a clear therapeutic advantage of using a related versus unrelated donor (e.g., son or daughter versus friend or domestic partner), volunteer donors were recruited into the FMT program. The advantages of this change included removing the burden of donor identification from the patient, improving the efficiency and costs related to donor screening, a more consistent supply donor fecal microbiota, and the ability to impose extensive and stringent exclusion criteria on donor selection (Table 1). Two unpaid volunteer donors were recruited during this period, and one of them provided the majority of donated fecal material. Donor medical history was reviewed prior to every donation and complete laboratory screening, as described above, was done every 6 months.

TABLE 1
Donor exclusion criteria.

| Donor Exclusion Criteria | History and Physical Examination | Laboratory Screening |
|---|---|---|
| Risk of Infectious | 1. Known HIV or Hepatitis B, C | 1. Ab for HIV 1 and 2. |
| Agent | infection. | 2. Viral Hepatitis A IgM. |
| | 2. Known exposure to HIV or viral | 3. Hepatitis B surface Ag and core |
| | hepatitis at any time. | Ab. |
| | 3. High risk behaviors including sex | 4. HCV Ab. |
| | for drugs or money, men who have | 5. RPR. |
| | sex with men, more than one sexual | 6. Stool cultures for enteric pathogens |
| | partner in the preceding 12 months, | including Salmonella, Shigella, |
| | history of incarceration, any past use | Yersinia, Campylobacter, E. Coli |
| | of intravenous drugs or intranasal | 0157:H7. |
| | cocaine. | 7. Ova and parasites examination. |
| | 4. Tattoo or body piercing within 12 | 8. Positive stool Giardia, |
| | months. | Cryptosporidium and Helicobacter |
| | 5. Travel to areas of the world with | pylori antigens. |
| | increased risk of traveler's diarrhea. | 9. Clostridium difficile toxin B PCR. |
| | 6. Current communicable disease, e.g., | 10. Liver function tests including |
| | upper respiratory tract viral infection. | alkaline phosphatase, AST, ALT. |
| Gastrointestinal comorbidities | 1. History of irritable bowel | |
| | syndrome, or any of the associated | |
| | symptoms, including frequent | |
| | abdominal cramps, excessive gas, | |
| | bloating, abdominal distension, fecal | |
| | urgency, diarrhea or constipation. | |
| | 2. History of inflammatory bowel | |
| | disease such as Crohn's disease, | |
| | ulcerative colitis, lymphocytic colitis. | |
| | 3. Chronic diarrhea. | |
| | 4. Chronic constipation or use of | |
| | laxatives. | |
| | 5. History of gastrointestinal | |
| | malignancy or known colon polyposis. | |
| | 6. History of any abdominal surgery, | |
| | e.g., gastric bypass, intestinal | |
| | resection, appendectomy, | |
| | cholecystectomy, etc. | |
| | 7. Use of probiotics or any other over | |
| | the counter aids for specific purposes | |
| | of regulating digestion. | |
| Systemic Medical Conditions | 1. Established metabolic syndrome or | 1. Serum triglycerides (>150 mg/dL). |
| | any early features suggestive of its | 2. HDL cholesterol < 40 mg/dL |
| | emergence. Body mass index > 26 | (males) and <50 mg/dL (females). |
| | kg/m2, waste:hip ratio > 0.85 (male) | 3. High sensitivity CRP > 2.4 mg/L. |
| | and >0.8 (female); BP > 135 mmHg | 4. Fasting_plasma glucose > 100 |
| | systolic and >85 mmHg diastolic. 2. | mg/dL. |
| | Known systemic autoimmunity, e.g., | 5. Liver function tests, including |
| | connective tissue disease, multiple | alkaline phosphatase, AST, ALT. |
| | sclerosis, etc. | 6. FANA. |
| | 3. Known atopic diseases including | |
| | asthma or eczema. | |
| | 4. Chronic pain syndromes including | |
| | fibromyalgia, chronic fatigue | |
| | syndrome. | |
| | 5. Ongoing (even if intermittent) use | |
| | of any prescribed medications, | |
| | including inhalers or topical creams | |
| | and ointments. | |
| | 6. Neurologic, neurodevelopmental, | |
| | and neurodegenerative disorders | |
| | including autism, Parkinson's disease, | |
| | etc. | |
| | 7. Presence of a skin rash, wheezing on | |
| | auscultation, lymphadenopathy, | |
| | hepatomegaly or any stigmata of liver | |
| | disease, swollen or tender joints, | |
| | muscle weakness, abnormal | |
| | neurological examination. | |
| Additional factors known to affect | 1. Antibiotics for any indication within | |
| the composition of intestinal | the preceding 6 months. | |
| microbiota | | |

### Donor Material Preparation

Individual patient-identified donors used in the early phase of the program came into the outpatient endoscopy center 1-2 h prior to the scheduled procedure. The fecal material was collected in a toilet hat and processed in a dedicated bathroom separate from the procedure room. Approximately 50 gm of fecal material was placed into a standard commercial blender (Oster, Subeam Corp, Rye, N.Y.) and homogenized in 250 mL of sterile, nonbacteriostatic normal saline. The slurry was then passed through stainless steel tea strainers to remove larger particles that could interfere with loading the syringes.

The material obtained from volunteer “universal” donors was transported on ice into the laboratory, where it was processed within two hours of collection. The material was weighed and homogenized in a commercial blender in a dedicated biological cabinet. The slurry was then passed through 2.0 mm, 1.0 mm, 0.5 mm, and 0.25 mm stainless steel laboratory sieves (W. S. Tyler, Inc., Mentor, Ohio) to remove undigested food and smaller particulate material. The resulting material passing through the 0.25 mm sieve was centrifuged at 6,000×g for 15 min in a Sorvall SS-34 rotor and resuspended to one half the original volume in nonbacteriostatic normal saline. The resulting concentrated fecal bacteria suspension was administered to the patient immediately or amended with sterile pharmaceutical grade glycerol (Sigma, St. Louis, Mo.) to a final concentration of 10%, and stored frozen at −80° C. for one to eight weeks until used. Thawing was done over 2-4 hours in an ice bath prior to the FMT procedure. The frozen preparation was diluted to 250 ml with nonbacteriostatic normal saline prior to infusion in the donor. This fecal material extract, whether fresh or frozen, was nearly odorless and of reduced viscosity, color, and texture relative to earlier material prepared in the endoscopy center. Filtration of donor material allowed for effortless loading of large tip 60 mL syringes without risk of clogging. All containers, bottles, and sieves used in material preparation were sterilized prior to use. Fecal material from universal donors was treated in the same manner as that obtained from patient-identified donors.

### Transplantation Procedure

Patients were maintained on full dose of vancomycin (125 mg, 4 times daily, by mouth) until two days prior to the FMT procedure. The day before the procedure the patients were prepped using a split dosage polyethylene glycol purge (GoLYTELY or MoviPrep), which is standard in our endoscopy unit, prior to colonoscopies to wash out residual antibiotic and fecal material. The patients underwent a full colonoscopy under conscious sedation. Mucosal biopsies were taken to rule out lymphocytic colitis in absence of obvious inflammatory bowel disease. The majority of the prepared donor material (220-240 mL) was administered via the colonoscope's biopsy channel into the patient's terminal ileum and cecum. In some cases, however, a small portion (50 mL) was also instilled into colonic areas containing maximal diverticulosis. Recovery procedure was identical to that routinely used for standard colonoscopy patients. All patients were instructed to contact the endoscopist in case of symptom recurrence, were formally followed in clinic 1-2 months after the procedure. Clearance of CDI was defined by resolution of diarrhea and negative stool testing for C. difficile at 2 months following FMT. All patients in this protocol also participated in a study examining fecal bacterial community structure, which involved collection of fecal specimens on days 3, 7, 14 and 1, 3, 6, and 12 months after the procedure. The research staff collected these specimens from the patient's places of residence, providing additional opportunities for symptom follow-up.

### Statistical Analysis

Non-categorical data were compared using unpaired Student's t-test. Categorical data were compared using Fisher's exact test. GraphPad Prism software was used to calculate two-tailed and two-sided p-values that were calculated with each test, respectively.

### Results

### Demographics

The group of patients with recurrent CDI described in this report clearly had refractory disease as evidenced by the average number of sequential relapses and duration of the condition (Table 2). Furthermore, many patients had multiple risk factors for high probability of recurrence, such as history of severe CDI as evidenced by hospitalization, frequent use of non-C. difficile intercurrent antibiotics, and advanced age (Hu et al. Gastroenterology 2009; 136:1206-14). All patients failed a long taper or pulsed regimen of vancomycin, and 40% of patients also failed an additional long course of vancomycin followed by a two-week rifaximin “chaser” regimen. One of these patients also failed a 4-week course of rifaximin. Several patients (3/43) took 2-4 week course of nita7oxanide, which also failed to clear the infection. Patients with inflammatory bowel disease were not excluded from the protocol. Thirty five percent of our patients (14 of 40) had underlying IBD, including Crohn's disease (6/14), ulcerative colitis (4/14), and lymphocytic colitis (4/14). The patients with IBD were generally younger (Table 3), but did not differ in the refractory nature of CDI or severity of presentation than older patients. However, the majority of patients without underlying IBD had moderate to severe diverticulosis.

TABLE 2
Demographics of patient population. The first 10 cases were done using patient-identified individual donors. After that, the protocol
shifted to use of a standard donor. Fresh material was used in the earlier cases, and later practice shifted to use of frozen material.

| | | | Duration | | | | | | | | |
|---|---|---|---|---|---|---|---|---|---|---|---|
| | | | (months) | Number of | | | | | | | |
| | | | of RCDI | Relapses | History of | | | | | | |
| Donor | Age | Female | (Mean ± | (Mean ± | Hospitalization | Interim | | | | | Success |
| Material | (Mean ± SD) | Gender | SD) | SD) | for CDI | Antibiotics | PPI | CRI | IBD | Diverticulosis | Rate |
| Individual Donor | 61 ± 22 | 70% | 12.7 ± 7.3 | 6.2 ± 3.0 | 70% | 60% | 60% | 30% | 30% | 50% | 7/10 |
| (n = 10) | | | | | | | | | | | (70%) |
| Standard Donor, | 55 ± 22 | 83% | 13.1 ± 9.8 | 6.4 ± 3.3 | 75% | 42% | 33% | 25% | 50% | 50% | 11/12 |
| Fresh Material | | | | | | | | | | | (92%) |
| (n = 12) | | | | | | | | | | | |
| Standard Donor, | 59 ± 21 | 67% | 10.1 ± 10.0 | 5.2 ± 3.0 | 38% | 43% | 43% | 14% | 24% | 48% | 19/21 |
| Frozen Material | | | | | | | | | | | (90%) |
| (n = 12) | | | | | | | | | | | |
| Total Experience | 59 ± 21 | 72% | 10.3 | 5.9 ± 3.3 | 56% | 48% | 47% | 21% | 33% | 49% | 37/43 |
| | | | | | | | | | | | (86%) |

RCDI = Recurrent C. difficile Infection
PPI = Proton Pump Inhibitor medication
CRI = Chronic Renal Insufficiency or Failure
IBD = Inflammatory Bowel Disease

TABLE 3
Comparison of patients without and with underlying IBD. Definition
of IBD includes patients with Crohn's disease, ulcerative colitis,
and incidentally discovered lymphocytic colitis.

| | Non-IBD | IBD | |
|---|---|---|---|
| | (n = 29) | (n = 14) | p Value |
| Age (Mean ± SEM) | 64.7 ± 3.3 | 44.6 ± 5.8 | p = 0.0021 |
| Female | 69% | 79% | P = 0.43 |
| | | | (NS) |
| Duration of RCDI | 13.5 ± 2.1 | 8.3 ± 3.3 | 0 = 0.09 |
| (Mean # of | | | (NS) |
| months ± SD) | | | |
| Number of Relapses ± SD | 6.2 ± 3.0 | 4.4 ± 1.3 | p = 0.04 |
| | | | p = 1.00 |
| Rate of Hospitalization | 55% | 57% | P = 100 |
| | | | (NS) |
| Interim Antibiotics | 51% | 36% | p = 0.35 |
| | | | (NS) |
| PPI | 48% | 43% | p = 1.00 |
| | | | (NS) |
| Renal Insuficiency | 32% | 14% | p = 0.69 NS) |
| Diverticulosis | 69% | 14% | p = 0.0028 |

### Response to Treatment

The overall rate of infection clearance was 86% in response to a single infusion of donor fecal material, as evidenced by symptom resolution and negative PCR testing for C. difficile toxin B after two months of follow-up (Table 2). Negative testing for C. difficile toxin B for two months was accepted as therapeutic success in patients with underlying IBD, even in absence of complete symptom resolution. Three of ten patients (30%) who received FMT using material from patient-identified individual donors had a recurrence of CDI. Two standard donors were employed for the remaining 33 cases in this series, but the majority (30/33) were done using material prepared from a single donor. Three of 33 patients who received FMT from a standard donor (fresh or frozen) had a recurrence of CDI. The difference in donor source, patient-identified versus standard, was not significant (p=0.1270). There was no significant difference in clearing the infection with fresh (11/12) or frozen (19/21) donor material. All 6 patients who experienced recurrence of CDI after FMT were offered a repeat procedure. Two of these patients, both >80 years of age, had multiple other active medical problems and preferred to remain on indefinite treatment with vancomycin. Four other patients were treated with a second infusion, and all cleared the infection bringing the overall success rate to 95% (41 of 43 patients). All second infusions were performed using the standard donor derived material. One of the recurrences of CDI occurred in a patient who received his first infusion from the second standard donor. The same donor source was used for his second FMT. Three of the four patients who received a second FMT had underlying MD; two patients had Crohn's disease and one had lymphocytic colitis. Finally, the fourth patient had a partial colon resection done for a stricture that developed following her initial CDI episode. She has a colostomy draining her proximal colon and a long segment of residual distal colon. After recurrence of CDI within three weeks following her first FMT we thought it was likely that engraftment in this case was complicated by difficulty in retaining the donor material due to high flow of fecal contents and relatively small size of the infected colon. The second infusion in this case was done with two doses of frozen standard donor material: one via the colostomy into the colon and the other into the jejunum using upper push enteroscopy. C. difficile testing of her fecal material was done weekly in the first month and monthly thereafter. No C. difficile was found over three months of follow-up.

No serious adverse events were noted following FMT in any of the patients, with ether fresh or frozen materials. A minority of patients (approximately a third) noted some irregularity of bowel movements and excessive flatulence during the first couple weeks following the procedure, but these symptoms resolved by the time they were seen in clinic follow-up. Enhanced colitis activity in patients with underlying IBD was not observed and there was improvement in overall colitis activity in all patients with UC, although that is easily attributable to clearing the CDI. Interestingly, all diagnoses of lymphocytic colitis were made for the first time from biopsies taken during the colonoscopies performed at the time of FMT. These patients completely normalized their bowel function and had no diarrhea after FMT without any additional medical therapy for lymphocytic colitis. Follow-up biopsies were not performed in these patients when they became asymptomatic.

### Discussion

Recurrent infection is one of the most difficult clinical challenges in the spectrum of C. difficile induced diarrheal disease. The risk of recurrence increases up to 65% after two or more episodes (McDonald et al. Emerg Infect Dis 2006; 12:409-15), and this risk is nearly certain in older patients who suffered severe CDI and suffered additional disruption of gut microbiota from intercurrent administration of non-C. difficile suppressing antibiotics (Hu et al. Gastroenterology 2009; 136:1206-14). The inclusion criteria for patients in this case series were simple: at least three recurrences and failure of standard antibiotic treatments. Our patients averaged about six recurrences over an average course of one year. This population highlights known risk factors for recurrence of CDI other than documented recurrence. The majority had history of at least one hospitalization for severe CDI and almost half took antibiotics after developing CDI for another non-C. difficile indication. Patients with inflammatory bowel disease dominated the younger age group. Virtually all patients were taking probiotics at presentation and many have also tried toxin-binding resins. We did not systematically collect information on all the various probiotics preparations taken by our patients, and many have tried multiple types through the course of their recurrent infections. The most common preparations contained Saccharomyces boulardii and strains of Lactobacilli. All patients were recommended to discontinue taking probiotics after FMT. In summary, by all available indicators the patients in this case series had recalcitrant CDI that would not have had a significant response rate to a placebo, and were unlikely to respond to another course of antibiotics or other available therapeutic options.

FMT has been used for decades as a last ditch method to cure recurrent CDI, and there has been growing uncontrolled evidence supporting its efficacy. Here we report one of the largest single case series. The 95% overall success rate in this series is comparable to the cumulative experience in the literature (Bakken. Anaerobe 2009; 15:285-9, van Nood et al. Euro Surveill 2009; 14, Khoruts and Sadowsky. Mucosal Immunol 2011; 4:4-7), and adds to the impetus for developing this therapeutic approach to make it more widely available. The major issues tackled by our center were those of practicality. In the early phase of the program we asked the patients to bring in prospective donors, which is the most common approach in practice at this time. Our experience does not contradict the efficacy of this approach. However, donor identification and work-up increased expense of the procedure and introduced a potential delay period. Moreover, some patients who were already exhausted by the illness had difficulty in finding suitable donors. While the ideal state of donor health may not be essential for elderly recipients with limited life expectancy, we felt compromise was not an option for younger patients on any of the donor exclusion criteria. Gut microbiota constitute a human microbial organ with major functions in energy metabolism and function of the immune system (Khoruts and Sadowsky. Mucosal Immunol 2011; 4:4-7). Therefore, this transplant procedure has potential implications for systemic physiology of the recipient. While donor health is not a guarantee to optimal composition of gut microbiota, it is currently the only available indicator. For all these reasons we decided to introduce the standard donor option to our patients. Interestingly, although many patients came into clinic with some potential donor already identified, they all immediately preferred the standard option of an anonymous screened donor upon learning about it.

The next challenge became advance preparation of the donor material. Little is known about viability of different constituents of fecal microbiota over time, and we did not wish to test this variable. However, since production of fresh material on demand is not always practical, and does create delay and issues of sanitation and aesthetics, we introduced frozen donor material as another treatment option. The clinical efficacy of frozen preparation became quickly evident and it has now become part of the standard protocol in our program.

FMT is typically considered a last choice, desperate therapy option by most clinicians, and to a great extent that is due to multiple aesthetic and practical barriers that stand in the way of its administration. Increased prevalence, morbidity, and mortality of CDI has now reached epidemic proportions and a significant fraction of these patients cannot clear the infection with standard therapies. These patients may benefit from FMT, but it is likely that the procedure is not available to them. Our FMT protocol has now progressed to the point where most obvious aesthetic and practical challenges have been overcome. This also significantly reduces costs associated with screening of potential donors. While effort and organization is required for recruitment and screening of suitable donors, as well as material preparation and banking, execution of actual FMT has become a simple matter of loading the syringes with thawed, nearly odorless, material and a colonoscopy.

There are a number of limitations to this study. It was not a rigorous clinical trial designed to test efficacy of a particular FMT methodology versus another, or some other fowl of therapy. Instead, it was an attempt to standardize FMT, as the procedure protocol evolved in the course of our clinical experience. Additional work is needed to ready this procedure for clinical trials and wider application. Nevertheless, our clinical outcomes provide very convincing evidence for efficacy of the frozen preparations. However, we cannot conclude from this experience alone that the fresh and frozen preparations are equivalent. The complexity of the donor material preparations, technical inability to culture most of the contained microbial constituents by classic laboratory techniques, and our ignorance as to the identity of species that are therapeutically most important precluded simple tests of donor material prior to FMT that could predict its efficacy. However, we are currently working to characterize the microbial composition of donor material and recipients' fecal samples collected over time by high throughput 16S rRNA gene sequencing. Results of these experiments should provide some means to compare different donor preparations. In addition, we are working to develop practical laboratory tests that will allow for further standardization of microbial composition of donor preparations.

While application of FMT for recurrent CDI has a long history, case reports suggest that it may also have a place in treatment of MD and IBS (Bennet et al. Lancet 1989; 1:164, Borody et al. J Clin Gastroenterol 2003; 37:42-7, Andrews and Borody. Med J Aust 1993; 159:633-4). Given the potentially important role of gut microbiota in pathogenesis of the metabolic syndrome, FMT is already being explored in a clinical trial for this condition (Vrieze et al. Diabetologia 2010; 53:606-13). Simplification and standardization of FMT-based therapeutics is critical for its future development. Recent technological advances have also made it possible to gain insight into composition of gut microbiota and their activity. The study of microbiota in the context of FMT should accelerate development of microbial therapeutics and yield new insights into microbial host interactions.

### Example 5

### Production of Freeze Dried Fecal Microbiota for Fecal Microbiota Transplantation (FMT)

All processing steps were completed in a Class 2 biological safety cabinet. The processing equipment container (that was eventually used to place blenders and sieves that come into contact with feces) was filled with a 10% bleach solution. The donor fecal material was removed from a transport container. Donor fecal material was transferred to a sterile weighing container using a sterile tongue depressor. Up to three multiples of 50 grams of fecal material were weighed for a final weight of 150 grams and transferred to a sterile blending chamber. Using a sterile graduated cylinder, 250 ml of sterile phosphate buffered saline (PBS) was added for every 50 grams of fecal material, up to a total of 750 ml. The lid was secured onto the blending chamber and mounted on a blender drive unit.

A nitrogen gas hose was attached to the tubing connector on the blender lid. An outflow tube was attached to the other connection on the blender lid, and the outflow hose was attached to a vacuum flask. The gas valve on the nitrogen tank was opened and set to a flow rate of 1.5 liters/min and the blending chamber purged for 3 minutes. The nitrogen gas valve was left open while blending. The sample was blended three times, each for 20 seconds, with a 10 second pause between blending.

A sterilized sieve stack containing 2.0, 1.0, 0.5, and 0.25 mm sieves was assembled from top to bottom, respectively. The collection pan was at the bottom. The blended fecal slurry was poured on the 2.0 mm sieve at the top of stack. As much of the sieve surface area as possible was covered to prevent clogging. The material was allowed to pass through the 2.0 mm sieve, and the filter stack was tilted as necessary to allow material to run through the sieve. The 2.0 mm sieve was removed from the filter stack and held over the stack at an angle to allow material on the bottom of the sieve to run onto the sieve stack. The material was allowed to pass through the 1.0 mm sieve, and the filter stack was tilted as necessary to allow material to run through the sieve. The 1.0 mm sieve was removed from the filter stack and held over the stack at an angle to allow material on the bottom of the sieve to run onto the sieve stack. The same procedure was followed for the 0.5 mm sieve and the 0.25 mm sieve, and the final material, the intermediate fecal slurry, collected in the collection pan.

The intermediate fecal slurry from the collection pan was transferred to a sterile 250 ml centrifuge bottle using sterile conditions. The approximate volume in each bottle was recorded, and centrifuged for 15 minutes at 4500 rpm in GSA rotor at 4° C.

The bottles were removed from the centrifuge and the supernatant discarded. Sterile PBS was added to one half the original volume in each centrifuge bottle and the pellet resuspended. Sterile PBS was added to the original volume, and the bottles centrifuged for 15 minutes at 4500 rpm at 4° C. The supernatants were discarded.

Approximately one third of the original volume sterile PBS was added to each centrifuge bottle and the pellet resuspended. The washed material was combined into a single centrifuge bottle and mixed to ensure the slurry was well mixed. The slurry was transferred to a sterile 250 ml graduated cylinder, and transfer of large unsuspended particles from the centrifuge bottle to the cylinder was avoided. The volume of the slurry was recorded and transferred into a new 250 ml centrifuge bottle.

One ml of the fecal bacteria slurry was removed from the centrifuge bottle and transferred to a microfuge tube for microscopy and total protein assay, and stored in a refrigerator. Total protein measured by boiling preparations followed by the BCA assay. Two sets of serial dilutions of the fecal bacterial slurry were prepared in sterile saline (0.85% NaCl). The sample was diluted to 1:10, 1:100, 1:1000, and 1:10000, and a microbial cell count performed using a hemocytometer.

The volume of the intermediate fecal slurry necessary to provide 2.5×10¹² total microbes was calculated and the appropriate volume of the intermediate fecal slurry was transferred into a 100 ml bottle. An equal volume of 20% mannitol was added, to give a final mannitol concentration of 10%. After mixing, the suspension was transferred to a freeze drying jar which was then covered. A dry ice/ethanol bath was used to rapidly freeze the sample while holding the freeze drying jar at an angle and rotating it by hand in the dry ice/ethanol bath so that the fecal bacteria suspension froze in a thin layer on the sides of the jar.

The frozen suspension in the jar was stored at −80° C. until starting the freeze drying process. A freeze dryer was used for 24-48 hours to completely dry the material. After the material was completely dry, the freeze drying jar was removed from the freeze drying machine and transferred to a BSL-2 safety hood to protect from aerosolization of the freeze dried material. A sterile scoop was used to scrape the freeze dried material off the walls of the jar and break up large chunks of the material. The freeze dried material was transferred to a disinfected bone grinder cup, and ground with three 10 second pulses. The resulting material was a fine powder. The powdered material was transferred to a 100 ml storage container and stored at −80° C. until use.

### Example 6

Clinical Case Histories of Two Patients Treated with Fecal Microbiota Prepared on the Same Day in Liquid Frozen Form and Freeze-Dried Form.

Patient 1. An 83 year old woman presented with 8 month history of multiply recurrent C. difficile infection. The infection was originally triggered by urinary sepsis for which she was hospitalized. She was initially treated for the C. difficile infection with metronidazole, but on recurrence was treated with vancomycin 125 mg orally 4×/day for two weeks. She had another recurrence within 1-2 weeks after completing the course of vancomycin. She was then retreated with vancomycin, but placed on an extended taper course lasting approximately 8 weeks. She had relapsed again after completing that course and stayed on vancomycin until her evaluation for fecal microbiota transplantation with us. She continued to have residual diarrheal symptoms while on vancomycin of loose bowel movements.

Fecal Microbiota Tranpslantation. The patient continued on vancomycin until 2 days before the procedure. One day before the procedure she took Moviprep to cleanse her colon and wash out any residual antibiotics. One unit of donor fecal microbiota (2.5×10¹² bacteria), frozen in 10% glycerin, was thawed in an ice bath for two hours and resuspended in room temperature non-bacteristatic, sterile normal saline to 240 mL. This material was injected via the biopsy port of a colonoscope in the cecum. Colonoscopy was otherwise unremarkable.

The patient's diarrhea symptoms have resolved over several weeks and she has not had recurrence of C. difficile infection for subsequent six months of follow-up.

Patient 2. A 78 year old woman presented to us after 3 month treatment with vancomycin for C. difficile infection. The infection was acquired in the hospital following back surgery and necessitated an extended hospital stay (8 days for the C. difficile infection itself). She continued to have diarrheal symptoms despite being on vancomycin. The vancomycin was tapered off, but she reported increased diarrhea and was placed fidaxomicin 200 mg twice daily. The patient continued to have diarrheal symptoms of about 8 times per day despite being on this antibiotic. After completion of fidaxomicin she was once again documented to be C. difficile positive with ongoing diarrhea. She was placed on vancomycin.

Fecal Microbiota Transplantation. The patient continued on vancomycin until 2 days before the procedure. One day before the procedure she took GoLytely to cleanse her colon and wash out any residual antibiotics. One unit of donor freeze-dried fecal microbiota (2.5×10¹² bacteria) was suspended in 240 mL of non-bacteriostatic, sterile normal saline. This material was injected via the biopsy port of a colonoscope into the terminal ileum and the cecum. The examination was otherwise notable only for mild diverticulosis.

The patient had resolution of her symptoms over the next several weeks and had not had a recurrence of C. difficile infection in subsequent 5 months of follow-up.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A freeze-dried composition comprising an extract or preparation of human feces, wherein the composition comprises:
biological material and a cryoprotectant,
wherein the freeze-dried composition is friable, wherein optionally the composition comprises a pharmaceutically acceptable carrier, and optionally the composition is a formulation for oral administration,
and wherein the freeze-dried composition, upon reconstitution with water, comprises no greater than about 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% weight non-living material/weight biological material, wherein the biological material comprises human gut, colon or intestinal fecal microbes, and optionally the biological material comprises human gut, colon or intestinal bacteria.

2. (canceled)

3. (canceled)

4. (canceled)

5. The freeze-dried composition of claim 1 wherein the cryoprotectant comprises skim milk, gelatin, mannitol, or a combination thereof.

6. The freeze-dried composition of claim 1 wherein the composition comprises at least two cryoprotectants, wherein the first cryoprotectant is sucrose.

7. The freeze-dried composition of claim 6 wherein the second cryoprotectant is selected from skim milk, gelatin, and mannitol.

8. The freeze-dried composition of claim 1, wherein the cryoprotectant is present at a concentration of at least 1%, 2%, 3%, 4%, 5%, 6%, 7%, 8%, 9%, or 10% (vol/vol).

9. (canceled)

10. The composition of claim 1 wherein the composition comprises at least about 1×10¹², 1.5×10¹², 2×10¹², or 2.5×10¹² bacteria.

11. The composition of any of claims 1 or wherein the biological material comprises: a plurality of prokaryotic cells, eukaryotic cells, or viruses; or a population of prokaryotic cells, eukaryotic cells, and viruses, that is substantially identical to or representative of or equivalent to a population of prokaryotic cells, eukaryotic cells, and viruses present in gut, intestine, colon, or feces of a noimal healthy human.

12. The composition of claim any of claims 1 wherein the biological material present comprises a population of prokaryotic cells and viruses that is substantially identical to or representative of or equivalent to a population of prokaryotic cells and viruses present in the feces of a normal healthy human.

13. (canceled)

14. A composition prepared by a process comprising:
subjecting a fecal sample to a condition or conditions that remove at least about 91%, 92%, 93%, 94% 95%, 96%, 97%, 98%, 99% or more of the non-living material present in the fecal sample prior to the subjecting to result in an extract;
adding a cryoprotectant to the extract to result in a mixture; and
freeze-drying the mixture to result in the composition,
wherein the composition comprises a biological material, and optionally the biological material comprises bacteria,
wherein optionally the composition comprises a pharmaceutically acceptable carrier, and optionally the composition is a formulation for oral administration.

15. The composition of claim 14 wherein the subjecting occurs at a temperature of no greater than about 26° C., 27° C., 28° C., 29° C., 30° C., 31° C., 32° C., 33° C., or 34° C.

16. (canceled)

17. The composition of claim 14 wherein the process further comprises reconstituting the composition with an aqueous solution.

18. (canceled)

19. (canceled)

20. The composition of claim 14 wherein the composition comprises at least 4 different phyla of bacteria,
wherein the phyla comprise a Bacteroidetes, a Firmicutes, a Proteobacteria, a Tenericutes phyla, or a combination thereof,
wherein optionally the phyla are chosen from Bacteroidetes, Firmicutes, Proteobacteria, Tenericutes, or a combination thereof.

21. The composition of claim 20 wherein the composition further comprises at least 5, 6, 7, 8, 9, or 10 different classes of bacteria chosen from Actinobacteria, Bacteroidia, Bacilli, Clostridia, Erysipelotrichi, Alphaproteobacteria, Betaproteobacteria, Gammaproteobacteria, Mollicutes, and Verrucomicrobiae.

22. The composition of claim 1 wherein the composition is encapsulated in a capsule.

23. The composition of claim 22 wherein the capsule comprises an acid-resistant enteric coating.

24. A composition as described in claim 1 for use as a therapeutic agent.

25. A composition as described in claim 1 for use in the treatment of a disease or a pathological or iatrogenic condition of the colon.

26. The composition of claim 25 wherein the disease is a disease or condition characterized by a dysfunctional or pathological composition of colon microbiota.

27. The composition of claim 26 wherein the disease is a Clostridium difficile colitis.

28. The method of claim 27 wherein the Clostridium difficile colitis is selected from an acute Clostridium difficile colitis, a relapsing Clostridium difficile colitis, and a severe Clostridium difficile colitis.

29.-44. (canceled)
